(19) United States
(12) Patent Application Publication
(10) Pub. No.: US 2021/0386743 A1
(43) Pub. Date: Dec. 16, 2021

(54) USE OF PDE9 INHIBITORS FOR TREATMENT

(71) Applicant: The Johns Hopkins University, Baltimore, MD (US)

(72) Inventors: David Kass, Columbia, MD (US); Dong Ik Lee, Baltimore, MD (US); Sumita Mishra, Baltimore, MD (US)

(21) Appl. No.: 17/283,217

(22) PCT Filed: Oct. 8, 2019
(86) PCT No.: PCT/US2019/055096

§ 371 Date: Apr. 6, 2021

Related U.S. Application Data
(60) Provisional application No. 62/742,674, filed on Oct. 8, 2018.

(51) Int. Cl. A61K 31/519; A61K 45/06

(52) U.S. Cl. CPC A61K 31/519; A61K 45/06

(57) ABSTRACT

Studies in female mice lacking ovaries (and thus estrogen), and placed on a high fat diet (60% fat) to induce severe obesity, and then stimulated with a low level of high pressure stress on the heart to induce mild hypertrophy and activate natriuretic peptide signaling. Unlike females on the same diet and heart stress but with their ovaries, those without ovaries demonstrated marked weight loss from phosphodiesterase E-9 (PDE9) inhibition, in combination with improvement in their metabolic signature (reduced fasting glucose, cholesterol, and triglycerides), without any change in food intake, nor change in activity. Thus, in one aspect, the invention provides methods for decreasing body fat and increasing lean muscle mass in estrogen deficient obese female subjects comprising administering to the subjects, at least one PDE9 inhibitor.

### REFERENCE TO RELATED APPLICATIONS

This application claims the benefit of U.S. Provisional Patent Application No. 62/742,674, filed on Oct. 8, 2018, which is hereby incorporated by reference for all purposes as if fully set forth herein.

### STATEMENT OF GOVERNMENTAL INTEREST

This invention was made with government support under grant no. R35 HL135827-01 awarded by the National Institutes of Health. The government has certain rights in the invention.

### BACKGROUND OF THE INVENTION

According to the World Health Organization, in 2016 there were approximately 1.9 billion overweight adults aged 18 years and older, of which 650 million were obese. Overall, obesity affects 13% of the world's population. This rate is much higher in the United States. According to the Centers for Disease Control, nearly 40% of Americans have a body mass index >30 (obesity), and nearly 8% have severe obesity (defined by a BMI >40). Obesity rates in Europe are lower, in the range of 15-20%, but this rate is rising.

Obesity is associated with multiple morbidities including type-2 diabetes mellitus, increased risk of cardiovascular disease including myocardial infarction, heart failure, and stroke, and increased risk of a number of cancers. While it is well known that decreasing calorie intake and increasing regular exercise are effective in combating obesity, this has been extremely difficult to achieve in practice. Part of this is the body's own capacity to regulate its metabolism so that even as food intake is reduced, metabolic rate declines so that weight is maintained. The development of alternative effective treatments for obesity has taken on increasing relevance and need.

One particularly notable syndrome now commonly associated with obesity and increasingly severe obesity is called heart failure with a preserved ejection fraction (HFpEF). HFpEF represents nearly half of all heart failure world-wide. Its distinction from heart failure with depressed cardiac function—or dilated cardiomyopathy—is that while the patient exhibits many of the same types of symptoms such as shortness of breath, fluid retention and tissue edema, fatigue and exertional limitations, the pump function of the heart appears to be essentially normal (e.g. preserved). At Johns Hopkins Hospital, the HFpEF population has a mean BMI in the upper 30's, with more and more patients presenting with severe obesity. A majority of affected patients are post-menopausal women. Importantly, to date no evidence-based therapies have proven successful in treating HFpEF.

One of the molecular pathways proposed to reduce fat stores by increasing lipolysis, enhancing glucose metabolism, reducing appetite, and enhancing the metabolic activity of fat, is associated with cyclic guanosine monophosphate (cGMP) and its activation of protein kinase G. Cyclic GMP is generated by one of two primary pathways and associated enzymes. The first pathway involves guanylyl cyclase 1 and 2 (GC-1, GC-2)—previously known as soluble guanylate cyclase. These proteins are activated by nitric oxide (NO) to produce cGMP, and so are coupled to intrinsic or extrinsic (e.g. pharmacological) stimulation of NO synthesis and its functionality. The pathway is coupled to receptor-couple cyclases GC-A and GC-B that are stimulated upon interaction of the receptor with natriuretic peptides. This links this pathway to levels of natriuretic peptides which are synthesized principally in heart muscle cells. These cyclase enzymes generate cGMP in different sub-cellular compartments, and their primary impact is to stimulate cGMP-dependent kinase (more commonly known as protein kinase G). PKG activation is best known for its role in reducing vascular smooth muscle tone, inducing systemic, coronary, pulmonary, and corpus cavernosal vasodilation. However, PKG stimulation by natriuretic peptides and nitric oxide signaling is also reported to counter excess fat accumulation in animal models of obesity and to enhance the metabolic activity of fat. In addition, PKG activation in the heart counters pathological fibrosis, hypertrophy, and improves heart function.

Among the methods used to stimulate PKG is the selective inhibition of members of the phosphodiesterase (PDE) enzymes that specifically degrade cGMP. There are three of these in the 11-member PDE superfamily, PDE6 being only expressed in the eye, while PDE5 and PDE9 (also known as PDE5A and PDE9A) are more broadly expressed in other tissues. Inhibition of PDE5 forms the basis for the current treatment of pulmonary hypertension and erectile dysfunction by drugs such as sildenafil. PDE9 is even more selective for cGMP, but until very recently, little was known about the pharmacology and therapeutic utility of selective phosphodiesterase-9 enzyme (PDE-9) inhibitors. The first of these agents were developed in the early 2000's (PDE9 was itself first reported in 1998), and once established as a cGMP-targeting PDE, various potential applications were envisioned. However, the only applications of PDE9 inhibitors that have been clinically pursued relate to its effects on cognitive function, as PDE9 was most highly expressed in areas of the brain, and for sickle-cell anemia. Clinical tests for use in Schizophrenia and Alzheimer's disease have been reported, neither applications have proven successful to date. Trials continue to test its role in sickle cell anemia. No studies have been reported on the impact of PDE9 inhibition on obesity or metabolism. In 2015, the Kass laboratory at Johns Hopkins (co-inventor) reported in Nature, (Lee et al, Nature, 519(7544): 472-6) that PDE9 is expressed at the protein level and is functionally important in the mammalian heart. Expression was demonstrated in human hearts and particularly in human heart failure, and it was first reported that PDE9 inhibition ameliorated stress-stimulated heart disease. In addition to these new discoveries, the 2015 landmark study revealed that PDE9 does not regulate cGMP generated by the NO-GC-1 or NO-GC-2 signaling pathway, but hydrolyzes cGMP generated by the GC-A and GC-B pathway. This is very important, since it meant that the efficacy of PDE9 inhibitors to treat heart disease was not diminished by conditions that blunt nitric oxide synthesis. By comparison, the only prior-known cGMP-selective PDE-PDE5—which is inhibited by drugs such as sildenafil, hydrolyzes cGMP derived from GC-1 and GC-2 linked to the NO-pathway, and its efficacy to ameliorate cardiac disease was lost in mice in which NO synthesis was suppressed.

There are several ways that the nitric oxide signaling pathway can be depressed. A major one is due to increased oxidative stress. This is common with aging, metabolic syndromes including obesity and type 1 and 2-diabetes; vascular disorders including hypertension, atherosclerosis, stiffening of the arteries; inflammatory diseases including viral, bacterial, and proteozoal infections; autoimmune diseases including rheumatological disorders and inflammatory bowel disease; environmental pollutants, smoking, and other disorders. In women, nitric oxide-related cGMP signaling naturally declines with menopause in association with the decline in estrogen.

Importantly, all existing studies regarding the impact of PKG stimulation on obesity and most all regarding its impact on heart disease, have studied male mammals only; no prior studies have reported on the influence of estrogen status on methods to stimulate PKG and impact obesity and associated metabolism. Specifically, the impact of estrogen status on the efficacy of PDE9 inhibition to counter obesity and associated diseases such as pressure-load heart disease, has not been previously tested. Thus, while the use of a PDE9 inhibitor to treat “obesity” was proposed in WO2005/041972, there was no insight at this time as which types of individuals this might apply to and why. Since this particular proposed use, there have been no published studies either confirming or refuting this application, likely because of the lack of such critical insight.

Broadly, there exists an unmet need to effectively treat obesity. In particular, methods that might do so in conditions in which nitric oxide signaling is deficient but natriuretic peptide signaling persists, including a major class—post-menopausal women, and also patient with obesity associated with HFpEF. These are two major populations for which there is no effective current therapy, and for which a targeted effective new treatment would have major impact.

### SUMMARY OF THE INVENTION

In accordance with an embodiment, the present invention provides methods for decreasing the percentage of body fat in a subject in which nitric oxide signaling is deficient, including subjects with diseases associated with oxidative stress, and in particular, estrogen deficient female subjects, comprising administering to the subject an effective amount of a phosphodiesterase-9 enzyme (PDE-9) inhibitor.

In accordance with an embodiment, the present invention provides methods for decreasing the percentage of body fat in a male subject in a subject in which nitric oxide signaling is deficient, including from a disease associated with oxidative stress, comprising administering to the subject an effective amount of a PDE-9 inhibitor.

In accordance with an embodiment, the present invention provides methods for decreasing the percentage of body fat in a female subject in which nitric oxide signaling is deficient, including from a disease associated with oxidative stress, comprising administering to the subject an effective amount of a PDE-9 inhibitor

In accordance with an embodiment, the present invention provides methods for decreasing the percentage of body fat in an estrogen deficient female subject, comprising administering to the subject an effective amount of a PDE-9 inhibitor.

In accordance with another embodiment, the present invention provides methods for increasing the percentage of lean muscle mass in a male subject in which nitric oxide signaling is deficient, including from a disease associated with oxidative stress, comprising administering to the subject an effective amount of a PDE-9 inhibitor.

In accordance with an embodiment, the present invention provides methods for increasing the percentage of lean muscle mass in a female subject in which nitric oxide signaling is deficient, including from a disease associated with oxidative stress, comprising administering to the subject an effective amount of a PDE-9 inhibitor.

In accordance with another embodiment, the present invention provides methods for increasing the percentage of lean muscle mass in an estrogen deficient female subject in which nitric oxide signaling is deficient, comprising administering to the subject an effective amount of a PDE-9 inhibitor.

In accordance with a further embodiment, the present invention provides methods for increasing the metabolic rate in a male subject in which nitric oxide signaling is deficient, including from a disease associated with oxidative stress, comprising administering to the subject an effective amount of a PDE-9 inhibitor.

In accordance with an embodiment, the present invention provides methods for increasing the metabolic rate in a female subject in which nitric oxide signaling is deficient, including from a disease associated with oxidative stress, comprising administering to the subject an effective amount of a PDE-9 inhibitor.

In accordance with a further embodiment, the present invention provides methods for increasing the metabolic rate in an estrogen deficient female subject in which nitric oxide signaling is deficient, comprising administering to the subject an effective amount of a PDE-9 inhibitor.

In accordance with an embodiment, the present invention provides methods for decreasing the body weight of a male subject in which nitric oxide signaling is deficient, including from a disease associated with oxidative stress, comprising administering to the subject an effective amount of a PDE-9 inhibitor.

In accordance with an embodiment, the present invention provides methods for decreasing the body weight in a female subject in which nitric oxide signaling is deficient, including from a disease associated with oxidative stress, comprising administering to the subject an effective amount of a PDE-9 inhibitor.

In accordance with an embodiment, the present invention provides methods for decreasing the body weight of an estrogen deficient female subject in which nitric oxide signaling is deficient, comprising administering to the subject an effective amount of a PDE-9 inhibitor.

In accordance with another embodiment, the present invention provides methods for decreasing cardiac hypertrophy in an obese a male subject in which nitric oxide signaling is deficient, including from a disease associated with oxidative stress, comprising administering to the subject an effective amount of a PDE-9 inhibitor.

In accordance with an embodiment, the present invention provides methods for decreasing cardiac hypertrophy in an obese female subject in which nitric oxide signaling is deficient, including from a disease associated with oxidative stress, comprising administering to the subject an effective amount of a PDE-9 inhibitor. In accordance with another embodiment, the present invention provides methods for decreasing cardiac hypertrophy in an obese estrogen deficient female subject in which nitric oxide signaling is deficient, comprising administering to the subject an effective amount of a PDE-9 inhibitor.

In accordance with a further embodiment, the present invention provides methods for improving cardiac function in an obese a male subject in which nitric oxide signaling is deficient, including from a disease associated with oxidative stress, comprising administering to the subject an effective amount of a PDE-9 inhibitor.

In accordance with an embodiment, the present invention provides methods for improving cardiac function in an obese female subject in which nitric oxide signaling is deficient, including from a disease associated with oxidative stress, comprising administering to the subject an effective amount of a PDE-9 inhibitor

In accordance with a further embodiment, the present invention provides methods for improving cardiac function in an obese estrogen deficient female subject in which nitric oxide signaling is deficient, comprising administering to the subject an effective amount of a PDE-9 inhibitor.

In accordance with yet another embodiment, the present invention provides methods for improving metabolic conditions, including increasing glucose tolerance and insulin sensitivity, and reducing hyperlipidemia, in a male subject in which nitric oxide signaling is deficient, including from a disease associated with oxidative stress, comprising administering to the subject an effective amount of a PDE-9 inhibitor.

In accordance with yet another embodiment, the present invention provides methods for improving metabolic conditions, including increasing glucose tolerance and insulin sensitivity, and reducing hyperlipidemia in an estrogen deficient female subject in which nitric oxide signaling is deficient, comprising administering to the subject an effective amount of a PDE-9 inhibitor.

In accordance with a further embodiment, the present invention provides methods for improving metabolic conditions, including increasing glucose tolerance and insulin sensitivity, and reducing hyperlipidemia in an obese estrogen deficient female subject in which nitric oxide signaling is deficient, comprising administering to the subject an effective amount of a PDE-9 inhibitor.

### BRIEF DESCRIPTION OF THE DRAWINGS

FIG. 1 is a diagram of an experimental protocol used to study morbid obesity in post-ovariectomized female mice, superimposed with mild cardiac stress induced by trans-aortic constriction (TAC), and then subsequently treated with a placebo control or active PDE-9 inhibitor.

FIG. 2 illustrates the generation of morbid obesity in female C57BL/6N mice. Mice are fed on a 60% high fat diet for a period exceeding 12 weeks. This strain shows notable obesity in female mice fed this diet. When both ovaries are further removed, weight gain is even more marked (over 2×normal weight), an equivalent of severe obesity.

FIGS. 3A-3E depict obese OVX+HFD female mice with nearly double their body weight versus OVX on normal chow (3A). The mice exhibit markedly abnormal insulin sensitivity, reflected by abnormal glucose (3B,C) and insulin (3D,E) tolerance test response curves.

FIGS. 4A-4C show OVX+HFD+TAC mice treated with either vehicle control or PDE9-inhibitor (PF 04447943). Inhibition of PDE9 leads to reduced left ventricular hypertrophy (LV Mass, 4A), and improved heart function reflected by greater fractional shortening (4B) and ejection fraction (4C). The arrow shows the time (1—week after TAC) when drug therapy was initiated.

FIG. 5 shows the end-of study data for cardiac size and systolic function, and for measures of diastolic function. Ventricular end-diastolic (Volume, d) and end-systolic (Volume, s) volumes, ejection fraction, cardiac output, isovolumic ventricular relaxation time (IVRT) and early to late atrial (E/A) filling are shown. OVX+HFD+TAC Mice treated with the PDE9 inhibitor show restoration towards normal control values for all these parameters compared vehicle treated mice.

FIGS. 6A-5E show the effects of PDE9 inhibition on body weight and lean and fat mass in OVX+HFD+TAC mice. (5A) Body weight declined by nearly 20% from PDE9 inhibitor treatment, a ˜30% relative weight reduction is compared to OVX mice on normal diet. Lean and fat body composition show a significant decline in fat weight (5B) that accounts for nearly all the weight loss, and no change in absolute lean body mass (5C). This results in reduced percent body fat (5D) and increased percent lean body mass (5E).

FIGS. 7A-6G show metabolic cage analysis for animal daily activity, food intake, respiratory exchange, and metabolism in mice with OVX+HFD+TAC treated with or without a PDE9 inhibitor. There is enhanced gas exchange (6A,B,C), with increases in oxygen consumption (6A) and carbon dioxide generation (6B). The respiratory exchange ratio (RER; 6C) was not significantly altered, and reflects primary fat consumption in the diet. Calculated energy expenditure (heat) increased (6D). This occurs without a concomitant change in food intake (6E), total activity (6F), or ambulatory activity (6G).

FIG. 8A shows end-of study data for total body weight, fasting blood glucose, fasting blood total cholesterol, and fasting total blood triglycerides. The latter three demonstrate metabolic syndrome induced in OVX+HFD+TAC mice, and each were reduced towards normal control levels by PDE9 inhibitor treatment. FIG. 8B shows histological sections of the liver from ovariectomized mice on a standard diet (OVX:normal diet), those on the HFD, and those on a HFD concomitantly treated with PDE9 inhibitor treatment. There is striking hepatic fat accumulation in those on the HFD with no additional therapy, whereas those on a HFD co-treated with the PDE9 inhibitor show marked reduction of fat accumulation in the liver.

FIG. 9A-9E shows fat and lean mass composition in C57BL6/N female mice with intact ovaries that were administered the identical HFD and then randomized to receive vehicle therapy or the PDE9 inhibitor. Unlike mice lacking their ovaries, these female mice did not have any alteration in total body weight, fat or lean mass, and corresponding percent fat or lean mass. Thus, the impact of PDE9 inhibition on body mass, and fat content requires a loss of sex hormone dependent signaling (e.g. OVX).

FIG. 10A-10G shows the results of overall oxygen consumption, CO₂ production, respiratory exchange ratio, calculated energy expenditure, food intake, total activity, and ambulatory activity in female mice with intact ovaries, fed a HFD, and subjected to pressure overload as outlined in the latter stages of the protocol in FIG. 1. Unlike female mice that had their ovaries removed, the female mice with intact ovaries do not show any changes in any of these metabolic parameters as a consequence of PDE9i treatment. Neither group of female mice +/−OVX show differences in food intake or activity from PDE9i treatment.

FIG. 11 illustrates an experimental protocol to study the effect of PDE9 inhibition in male mice provided a HFD and then exposed to pressure overload.

FIGS. 12A-12D show cardiac remodeling in obese male mice (HFD) subjected to trans-aortic constriction (TAC) to induce pressure overload stress. The PDE9 inhibitor significantly lowers cardiac hypertrophy (LV mass, 9A), and improves cardiac function (fractional shortening (9B), and ejection fraction (9C)). This response is analogous to what is observed in female OVX+HFD+TAC mice receiving the same PDE9 inhibitor. PDE9 inhibition also reduces cardiac hypertrophic induced by pressure overload and functional response to pressure-overload in vehicle treated HFD-males is quantitatively more than observed in OVX-HFD females, in part due to greater load imposed in males (somewhat larger aorta so bit tighter constriction). Natriuretic peptide expression in myocardium, a marker of pathological hypertrophy of the heart muscle, is reduced by PDE9-I therapy (9D).

FIG. 13 illustrates that male HFD-TAC mice display a trend towards reduced body weight, but also display a decline in body fat.

FIG. 14 shows that unlike OVX females on HFD and exposed to pressure overload (TAC), males did not display consistent changes in oxygen consumption, CO₂ generation, RER, energy expenditure.

### DETAILED DESCRIPTION OF THE INVENTION

The inventors' work has identified for the first time a subset of potential patients who might particularly benefit from PDE9 inhibition, those in which the NO-signaling pathway was compromised. Such patients would include those with inflammatory disorders such as accompanies severe obesity, diabetes, atherosclerosis and hyperlipidemias, and other organ diseases that activate oxidative stress. Oxidants chemically interact with NO to form other molecules that reduce the net concentration of NO needed to stimulate GC-1 or GC-2, and also depress the function of these two enzymes to respond to any NO that is generated. Thus, the net generation of cGMP and in turn activation of PKG is compromised.

Another major cause for reduced NO-dependent signaling occurs in older women after menopause. This is because in women, the decline in sex hormones, and particularly in estrogen after menopause results in impairment of NO signaling, as estrogen is a prominent stimulator of this pathway and its corresponding molecular changes. In premenopausal women, the estrogen pathway is responsible for protection against a variety of cardiovascular diseases that occurs more commonly in men at matched ages, and this has long been linked to enhanced NO signaling. However, after menopause, women develop similar risks to age-matched men for cardiovascular diseases, including coronary artery disease, stroke, myocardial infarction, heart-failure. Given this, one might anticipate that in females, loss of estrogen could also compromise the efficacy of a PDE5 inhibitor, which hydrolyzes (catabolizes) cGMP generated by the NO-GC-1 or NO-GC-2 pathway, to stimulate cGMP-PKG signaling. Takimoto et al. (JCI, 2014; 124:2464-71) tested this. Several different models of heart disease in female mice were successfully treated with sildenafil, a PDE5 inhibitor. However, when the ovaries were first removed, this treatment became totally ineffective. It was rescued by exogenous provision of estrogen replacement.

The fact that PDE9 targets the natriuretic peptide pathway means that activation of this pathway is important for efficacy. The fact that PDE9 can enhance PKG stimulation even if NO-GC-1/2 signaling is depressed, as occurs in women post-menopause was unknown, and so not studied. No studies have been reported (published report or abstracts) on the efficacy of PDE9 inhibitors to counter obesity. We believe this is likely due to highly variable results, with overall negative findings due to the underlying lack of understanding of the signaling involved, and that the efficacy of the inhibitor would not be apparent in many subjects lacking deficiency of nitric oxide signaling, elevated natriuretic peptides, and in particular in females, estrogen deficiency.

As the inventors now demonstrate in the present application, PDE9 inhibition is very effective when applied in the appropriate disease setting. Specifically, we performed studies in female mice lacking ovaries (and thus estrogen deprived), then placed on a high fat diet (60% fat) to induce severe obesity, and then further stimulated with a low level of high pressure-stress on the heart to induce mild hypertrophy and activate natriuretic peptide signaling. Unlike females on the same diet and heart stress but with their ovaries intact and so not estrogen deprived, those without ovaries (and estrogen) show marked weight loss from PDE9 inhibition, in combination with improvement in their metabolic signature (reduced fasting glucose, cholesterol, and triglycerides).

The weight loss occurs without any change in food intake, or change in activity, so the mechanism appears to engage intrinsic modulation of the fat that would otherwise be incorporated into body mass. We observe some benefit in male mice that always lack estrogen, though the magnitude is substantially less than observed in the ovariectomized females. Thus, the present invention, methods for using PDE9 inhibitors to treat obesity and associated co-morbidities including the heart in post-menopausal women (natural and iatrogenic) was not taught or suggested by any existing literature including published patent applications. Indeed, the failure of any publications subsequent to 2003 to even address the issue of PDE9 inhibition and obesity likely reflects the lack of meaningful findings prior to the current invention. The present invention was only possible once the inventors understood the preserved, if not enhanced, efficacy of PDE9 inhibition in conditions where the NO-GC signaling pathway is compromised.

In addition, the present inventors believe this inhibition will be most effective in conditions in which there is some enhanced synthesis of natriuretic peptide, due to the existence of cardiac disease. This makes the inventive methods particularly suitable for treatment of HFpEF. The present inventors also believe the inventive methods will be effective in disorders where the NO signaling pathway is compromised as this transfers additional physiological importance to the natriuretic peptide-signaling pathway that PDE9 regulates. In addition to women deficient in estrogen (post-menopause), we include conditions that stimulate oxidative stress that also compromise the NO-signaling pathway.

In addition, it will be understood by those of skill in the art that because males have very low levels of estrogen, in the context of the present invention, males can be considered to be estrogen deficient. The inventors believe that in males with diseases or other conditions that cause the NO signaling pathway to be compromised, administration of PDE-9 inhibitors will also provide similar metabolic effects, including decreased body fat, increased metabolism, and improved cardiac function.

In accordance with an embodiment, the present invention provides methods for decreasing the percentage of body fat in a subject in which nitric oxide signaling is deficient, including subjects with diseases associated with oxidative stress, and in particular, estrogen deficient female subjects, comprising administering to the subject an effective amount of a phosphodiesterase-9 enzyme (PDE-9) inhibitor.

In accordance with an embodiment, the present invention provides methods for decreasing the percentage of body fat in a male subject in a subject in which nitric oxide signaling is deficient, including from a disease associated with oxidative stress, comprising administering to the subject an effective amount of a PDE-9 inhibitor.

In accordance with an embodiment, the present invention provides methods for decreasing the percentage of body fat in a female subject in which nitric oxide signaling is deficient, including from a disease associated with oxidative stress, comprising administering to the subject an effective amount of a PDE-9 inhibitor

In accordance with an embodiment, the present invention provides methods for decreasing the percentage of body fat in an estrogen deficient female subject, comprising administering to the subject an effective amount of a PDE-9 inhibitor.

In accordance with another embodiment, the present invention provides methods for increasing the percentage of lean muscle mass in a male subject in which nitric oxide signaling is deficient, including from a disease associated with oxidative stress, comprising administering to the subject an effective amount of a PDE-9 inhibitor.

In accordance with an embodiment, the present invention provides methods for increasing the percentage of lean muscle mass in a female subject in which nitric oxide signaling is deficient, including from a disease associated with oxidative stress, comprising administering to the subject an effective amount of a PDE-9 inhibitor.

In accordance with another embodiment, the present invention provides methods for increasing the percentage of lean muscle mass in an estrogen deficient female subject in which nitric oxide signaling is deficient, comprising administering to the subject an effective amount of a PDE-9 inhibitor.

In accordance with a further embodiment, the present invention provides methods for increasing the metabolic rate in a male subject in which nitric oxide signaling is deficient, including from a disease associated with oxidative stress, comprising administering to the subject an effective amount of a PDE-9 inhibitor.

In accordance with an embodiment, the present invention provides methods for increasing the metabolic rate in a female subject in which nitric oxide signaling is deficient, including from a disease associated with oxidative stress, comprising administering to the subject an effective amount of a PDE-9 inhibitor.

In accordance with a further embodiment, the present invention provides methods for increasing the metabolic rate in an estrogen deficient female subject in which nitric oxide signaling is deficient, comprising administering to the subject an effective amount of a PDE-9 inhibitor.

In accordance with an embodiment, the present invention provides methods for decreasing the body weight of a male subject in which nitric oxide signaling is deficient, including from a disease associated with oxidative stress, comprising administering to the subject an effective amount of a PDE-9 inhibitor.

In accordance with an embodiment, the present invention provides methods for decreasing the body weight in a female subject in which nitric oxide signaling is deficient, including from a disease associated with oxidative stress, comprising administering to the subject an effective amount of a PDE-9 inhibitor.

In accordance with an embodiment, the present invention provides methods for decreasing the body weight of an estrogen deficient female subject in which nitric oxide signaling is deficient, comprising administering to the subject an effective amount of a PDE-9 inhibitor.

In accordance with another embodiment, the present invention provides methods for decreasing cardiac hypertrophy in an obese a male subject in which nitric oxide signaling is deficient, including from a disease associated with oxidative stress, comprising administering to the subject an effective amount of a PDE-9 inhibitor.

In accordance with an embodiment, the present invention provides methods for decreasing cardiac hypertrophy in an obese female subject in which nitric oxide signaling is deficient, including from a disease associated with oxidative stress, comprising administering to the subject an effective amount of a PDE-9 inhibitor. In accordance with another embodiment, the present invention provides methods for decreasing cardiac hypertrophy in an obese estrogen deficient female subject in which nitric oxide signaling is deficient, comprising administering to the subject an effective amount of a PDE-9 inhibitor.

In accordance with a further embodiment, the present invention provides methods for improving cardiac function in an obese a male subject in which nitric oxide signaling is deficient, including from a disease associated with oxidative stress, comprising administering to the subject an effective amount of a PDE-9 inhibitor.

In accordance with an embodiment, the present invention provides methods for improving cardiac function in an obese female subject in which nitric oxide signaling is deficient, including from a disease associated with oxidative stress, comprising administering to the subject an effective amount of a PDE-9 inhibitor

In accordance with a further embodiment, the present invention provides methods for improving cardiac function in an obese estrogen deficient female subject in which nitric oxide signaling is deficient, comprising administering to the subject an effective amount of a PDE-9 inhibitor.

In accordance with yet another embodiment, the present invention provides methods for improving metabolic conditions, including increasing glucose tolerance and insulin sensitivity, and reducing hyperlipidemia, in a male subject in which nitric oxide signaling is deficient, including from a disease associated with oxidative stress, comprising administering to the subject an effective amount of a PDE-9 inhibitor.

In accordance with yet another embodiment, the present invention provides methods for improving metabolic conditions, including increasing glucose tolerance and insulin sensitivity, and reducing hyperlipidemia in an estrogen deficient female subject in which nitric oxide signaling is deficient, comprising administering to the subject an effective amount of a PDE-9 inhibitor.

In accordance with a further embodiment, the present invention provides methods for improving metabolic conditions, including increasing glucose tolerance and insulin sensitivity, and reducing hyperlipidemia in an obese estrogen deficient female subject in which nitric oxide signaling is deficient, comprising administering to the subject an effective amount of a PDE-9 inhibitor.

In some or all of the above embodiments, the subject is a male subject in which the NO signaling pathway is deficient due to a disease or condition associated with oxidative stress.

In some or all of the above embodiments, the disease associated with oxidative stress is selected from the group consisting of: aging, metabolic syndromes including obesity and type 1 and 2-diabetes, vascular disorders including hypertension, atherosclerosis, stiffening of the arteries, inflammatory diseases including viral, bacterial, and protozoal infections; autoimmune diseases including rheumatological disorders and inflammatory bowel disease, environmental pollutants, smoking, and other disorders.

In some or all of the above embodiments, the subject is a female subject in which nitric oxide signaling is deficient, due to estrogen deficiency or menopause.

As used herein, the term “estrogen deficient female” means a female subject that no longer internally synthesizes estrogen in a clinically effective amount. In some embodiments, the female subjects can be menopausal or post-menopausal. In some other embodiments, the female subjects can have undergone bilateral oophorectomy alone or in combination with some other procedure (e.g. surgically induced menopause).

As used herein, the term “PDE9 inhibitor” is meant as an agent that reduces or attenuates the biological activity of the PDE9 (also known as PDE9A) polypeptide. Such agents may include proteins, such as anti-PDE9 antibodies, nucleic acids, e.g., PDE9 antisense or RNA interference (RNAi) nucleic acids, amino acids, peptides, carbohydrates, small molecules (organic or inorganic), or any other compound or composition which decreases the activity of a PDE9 polypeptide either by effectively reducing the amount of PDE9 present in a cell, or by decreasing the enzymatic activity of the PDE9 polypeptide. Compounds that are PDE9 inhibitors include all solvates, hydrates, pharmaceutically acceptable salts, tautomers, stereoisomers, and prodrugs of the compounds. Preferably, a small molecule PDE9 inhibitor used in the present invention has an IC₅₀ of less than 10 μM, more preferably, less than 1 μM, and, ever) more preferably, less than 0.1 μM. Any PDE9 inhibitor used in the present invention is preferably “selective” for PDE9 (PDE9A), such that it can be provided in effective pharmacological doses and not inhibit one or more of the other members of PDE superfamily: specifically PDE1A, PDE1B, PDE1C, PDE2A (PDE2), PDE3A, PDE3B, PDE4A, PDE4B, PDE4C, PDE4D, PDE5A (PDE5), PDE6, PDE7A, PDE7B, PDE8A, 5 PDE8B, PDE10, and/or PDE11.

As used herein, the term “selective” PDE9 inhibitor is meant as an agent that inhibits PDE9 activity with an IC₅₀ at least 10-fold less, preferably, at least 100-fold less, than the IC₅₀ for inhibition of one or more of the other PDEs. Preferably, such agents are combined with a pharmaceutically acceptable delivery vehicle or carrier. An antisense oligonucleotide directed to the PDE9 gene or mRNA to inhibit its expression is made according to standard techniques (see, e.g., Agrawal et al. Methods in Molecular Biology: Protocols for Oligonucleotides and Analogs, Vol. 20, 1993). Similarly, an RNA molecule that functions to reduce the production of PDE9 enzyme in a cell can be produced according to standard techniques known to those skilled in the art (see, e.g., Hannon, Nature 418: 244-251, 2002; Shi, Trends in Genetics 19: 9-12, 2003; Shuey et al., Drug Discovery Today 7: 1040-1046, 2002), J Med Chem. 2014 Dec. 26; 57(24): 10304-10313. Examples of PDE9 inhibitors are provided herein and in WO03/037899, in WO 03/037432, and U.S. patent application Ser. No. 10/828,485, filed Apr. 20, 2004 incorporated herein by reference.

Examples of such PDE9 inhibitors include, but are not limited to, 1-{[2-(3-isopropyl-7-oxo-6,7-dihydro-1H-pyrazolo[4,3-d]pyrimidin-5-ylmethyl)-phenoxy]-acetyl}-pyrrolidine-2-carbo-xylic acid; 1-{[2-(1-cyclopentyl-4-oxo-4,5-dihydro-1H-pyrazolo[3,4-d]pyrim-idin-6-ylmethyl)-phenox]l-acetyl}-pyrrolidine-2(S)-carboxylic acid 3-isopropyl-5-[2-(2-oxo-2-piperazin-1-yl-ethoxy)-benzyl]-1,6-dihydro-pyra-zolo[4,3-d]pyrimidin-7-one; 1-cyclopentyl-6-[2-(2-oxo-2-piperazin-1-yl-eth-oxy)-benzyl]-1,5-dihydro-pyrazolo[3,4-d]pyrimidin-4-one 3-isopropyl-5-[2-(2-morpholin-4-yl-2-oxo-ethoxy)-benzyl]-1,6-dihydro-pyra-zolo[4,3-d]pyrimidin-7-one; 3-isopropyl-5-[2-(2-oxo-2-pyrrolidin-1-yl-etho-xy)-benzyl]-1,6-dihydro-pyrazolo[4,3-d]pyrimidin-7-one; 5-{2-[2-(4-ethyl-piperazin-1-yl)-2-oxo-ethoxy]-benzyl}-3-isopropyl-1,6-di-hydro-pyrazolo [4,3-d]pyrimidin-7-one; N,N-diethyl-2-[2-(3-isopropyl-7-oxo-6,7-dihydro-1H-pyrazolo[4,3-d]pyrimidin-5-ylmethyl)-phenoxy]-acetamide; 1-{[2-(3-isopropyl-7-oxo-6,7-dihydro-1H-pyrazolo[4,3-d]pyrimidin-5-ylmethyl)-phenoxy]-acetyl}-pyrrolidine-2-carboxylic acid methyl ester; 4-{[2-(3-isopropyl-7-oxo-6,7-dihydro-1H-pyrazolo[4,3-d]pyrimidin-5-ylmeth-yl)-phenoxy]-acetyl}-piperazine-1-carboxylic acid tert-butyl ester; N-(2-dimethylamino-ethyl)-2-[2-(3-isopropyl-7-oxo-6,7-dihydro-1H-pyrazolo-[4,3-d]pyrimidin-5-ylmethyl)-phenoxy]-acetamide; 1-{[2-(1-cyclopentyl-4-ox-o-4,5-dihydro-1H-pyrazolo[3,4-d]pyrimidin-6-ylmethyl)-phenoxy]-acetyl}-pyr-rolidine-2-carboxylic acid methyl ester; 4-{[2-(1-cyclopentyl-4-oxo-4,5-dihydro-1H-pyrazolo[3,4-d]pyrimidin-6-ylmethyl)-phenoxy]-acetyl}-piperazine-1-carboxylic acid tert-butyl ester; 1-cyclopentyl-6-[2-(2-oxo-2-pyrrolidin-1-yl-ethoxy)-benzyl]-1,5-dihydro-pyrazolo[3,4-d]pyrimidin-4-one; 1-cyclopentyl-6-[2-(2-morpholin-4-yl-2-oxo-ethoxy)-benzyl]-1,5-dihydro-py-razolo[3,4-d]pyrimidin-4-one; 2-[2-(1-cyclopentyl-4-oxo-4,5-dihydro-1H-pyr-azolo[3,4-d]pyrimidin-6-ylmethyl)-phenoxy]-N-(2-dimethylamino-ethyl)-aceta-mide; 1-cyclopentyl-6-{2-[2-(4-ethyl-piperazin-1-yl)-2-oxo-ethoxy]-benzyl}-1,5-dihydro-pyrazolo[3,4-d]pyrimidin-4-one; 2-[2-(1-cyclopentyl-4-oxo-4,5-dihydro-1H-pyrazolo[3,4-d]pyrimidin-6-ylmethyl)-phenoxy]-N,N-diethyl-acet-amide; [2-(3-isopropyl-7-oxo-6,7-dihydro-1H-pyrazolo[4,3-d]pyrimidin-5-ylm-ethyl)-phenoxy]-acetic acid; [2-(1-cyclopentyl-4-oxo-4,5-dihydro-1H-pyrazo-lo[3,4-d]pyrimidin-6-ylmethyl)-phenoxy]-acetic acid; 3-isopropyl-5-[2-(5-chloro-2-morpholin-4-yl-ethoxy)-benzyl]-1,6-dihydro-pyrazolo[4,3-d]pyrimidin-7-one; 3-isopropyl-5-[2-(2-pyrrolidin-1-yl-ethoxy)-benzyl]-1,6-dihydro-pyrazolo[4,3-d]pyrimidin-7-one; 3-isopropyl-5-[2-(2-morpholin-4-yl-ethoxy)-cyclohexylmethyl]-1,6-dihydro-pyrazolo[4,3-d]pyrimidin-7-one; 5-[5-fluoro-2-(2-morpholin-4-yl-ethoxy)-be-nzyl]-3-isopropyl-1,6-dihydro-pyrazolo[4,3-d]pyrimidin-7-one; 3-cyclopentyl-5-[5-fluoro-2-(2-morpholin-4-yl-ethoxy)-benzyl]-1,6-dihydro-pyrazolo[4,3-d]pyrimidin-7-one; 3-isopropyl-5-[2-(2-morpholin-4-yl-ethoxy-)-benzyl]-1,6-dihydro-pyrazolo[4,3-d]pyrimidin-7-one; 9-(1,2-dimethyl-propyl)-2-[2-(2-morpholin-4-yl-ethoxy)-benzyl]-1,9-dihydr-o-purin-6-one; 2-[2-(2-morpholin-4-yl-ethoxy)-benzyl]-9-(tetrahydrofuran-3-yl)-1,9-dihydro-purin-6-one; 5-[2-(2-diethylamino-ethoxy)-benzyl]-3-isop-ropyl-1,6-dihydro-pyrazolo[4,3-d]pyrimidin-7-one; 3-cyclopentyl-5-[2-(2-mo-rpholin-4-yl-ethoxy)-benzyl]-1,6-dihydro-pyrazolo[4,3-d]pyrimidin-7-one; 3-cyclobutyl-5-[2-(2-morpholin-4-yl-ethoxy)-benzyl]-1,6-dihydro-pyrazolo[-4,3-d]pyrimidin-7-one; 9-(1(R),2-dimethyl propyl)-[2-(2-morpholin-4-yl-eth-oxy)-benzyl]-1,9-dihydro-purin-6-one; 9-(2-methyl-butyl)-2-[2-(2-morpholin-4-yl-ethoxy)-benzyl]-1,9-dihydro-purin-6-one; 9-cyclopentyl-2-[2-(2-morph-olin-4-yl-ethoxy)-benzyl]-1,9-dihydro-purin-6-one; 5-[2-(2-morpholin-4-yl-ethoxy)-benzyl]-3-pyridin-3-yl-1,6-dihydro-pyrazolo[4,3-d]pyrimidin-7-one; 9-(1,2-dimethyl-propyl)-2-[2-(2-morpholin-4-yl-ethoxy)-benzyl]-1,9-dihydr-o-purin-6-one; 9-isopropyl-2-[2-(2-morpholin-4-yl-ethoxy)-benzyl]-1,9-dihydro-purin-6-one; 2-[2-(2-morpholin-4-yl-ethoxy)-benzyl]-9-(tetrahydro-fura-n-2-ylmethyl)-1,9-dihydro-purin-6-one; 9-(1-isopropyl-2-methyl-propyl)-2-[-2-(2-morpholin-4-yl-ethoxy)-benzyl]-1,9-dihydro-purin-6-one; 9-(1-ethyl-propyl)-2-[2-(2-morpholin-4-yl-ethoxy)-benzyl]-1,9-dihydro-pur-in-6-one; 9-cyclopentyl-8-methyl-2[2-(2-morpholin-4-yl-ethoxy)-benzyl]-1,-1,-9-dihydro-purin-6-one; 3-cyclopentyl-5-[2-(2-morpholin-4-yl-ethoxy)-benzyl-]-3,6-dihydro-[1,2,3]triazolo[4,5-d]pyrimidin-7-one; 1-cyclopentyl-6-[2-(2-morpholin-4-yl-ethoxy)-benzyl]-1,5-dihydro-pyrazolo-[3,4-d]pyrimidin-4-one; 9-cyclopentyl-2-[2-(3-morpholin-4-yl-propoxy)-benz-yl]-1,9-dihydro-purin-6-one; N-[(1R,2S)2-(3-isopropyl-7-oxo-6,7-dihydro-1H-pyrazolo[4,3-d]pyrimidin-5-ylmethyl)-cyclohex-1-yl]-2-pyrrolidin-1-yl-acetamide; N-[(1R,2S)2-(3-isopropyl-7-oxo-6,7-dihydro-1H-pyrazolo[4,3-d]pyrim-idin-5-ylmethyl)-cyclohex-1-yl]-2-morpholin-4-yl-acetamide; 2-diethylamino-N-[(1R,2S)2-(3-isopropyl-7-oxo-6,7-dihydro-1H-pyrazolo[4,3-d]pyrimidin-5-ylmethyl)-cyclohex-1-yl]-acetamide; 1-{[(1R,2S)2-(3-isoprop-yl-7-oxo-6,7-dihydro-1H-pyrazolo[4,3-d]pyrimidin-5-ylmethyl)-cyclohex-1-yl-carbamoyl]-methyl}-pyrrolidine-2(S)-carboxylic acid methyl ester; 2-cyclobutylamino-N-[(1R,2S)2-(3-isopropyl-7-oxo-6,7-dihydro-1H-pyrazolo[-4,3-d]pyrimidin-5-ylmethyl)-cyclohex-1-yl]-acetamide; or 2-cyclopropylamino-N-[(1R,2S)2-(3-isopropyl-7-oxo-6,7-dihydro-1H-pyrazolo-[4,3-d]pyrimidin-5-ylmethyl)-cyclohex-1-yl]-acetamide; IMR-687, (a potent inhibitor of PDE9A), BAY73-6691, PF-04447943, PF-4181366, and stereoisomers, pharmaceutically acceptable salts, solvates or prodrugs thereof.

Pharmaceutically acceptable salts are art-recognized, and include relatively non-toxic, inorganic and organic acid addition salts of compositions of the present invention, including without limitation, therapeutic agents, excipients, other materials and the like. Examples of pharmaceutically acceptable salts include those derived from mineral acids, such as hydrochloric acid and sulfuric acid, and those derived from organic acids, such as ethanesulfonic acid, benzenesulfonic acid, p-toluenesulfonic acid, and the like. Examples of suitable inorganic bases for the formation of salts include the hydroxides, carbonates, and bicarbonates of ammonia, sodium, lithium, potassium, calcium, magnesium, aluminum, zinc and the like. Salts may also be formed with suitable organic bases, including those that are non-toxic and strong enough to form such salts. For purposes of illustration, the class of such organic bases may include mono-, di-, and trialkylamines, such as methylamine, dimethylamine, and triethylamine; mono-, di-, or trihydroxyalkylamines such as mono-, di-, and triethanolamine; amino acids, such as arginine and lysine; guanidine; N-methylglucosamine; N-methylglucamine; L-glutamine; N-methylpiperazine; morpholine; ethylenediamine; N-benzylphenthylamine; (trihydroxymethyl) aminoethane; and the like, see, for example, J. Pharm. Sci., 66: 1-19 (1977).

The term, “carrier,” refers to a diluent, adjuvant, excipient or vehicle with which the therapeutic is administered. Such physiological carriers can be sterile liquids, such as water and oils, including those of petroleum, animal, vegetable or synthetic origin, such as peanut oil, soybean oil, mineral oil, sesame oil and the like. Water is a suitable carrier when the pharmaceutical composition is administered intravenously. Saline solutions and aqueous dextrose and glycerol solutions also can be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene glycol, water, ethanol and the like. The composition, if desired, can also contain minor amounts of wetting or emulsifying agents, or pH buffering agents.

Further examples of biologically active agents include, without limitation, enzymes, receptor antagonists or agonists, hormones, growth factors, autogenous bone marrow, antibiotics, antimicrobial agents, and antibodies. The term “biologically active agent” is also intended to encompass various cell types and genes that can be incorporated into the compositions of the invention.

In certain embodiments, the subject compositions comprise about 1% to about 75% or more by weight of the total composition, alternatively about 2.5%, 5%, 10%, 20%, 30%, 40%, 50%, 60% or 70%, of a biologically active agent.

Non-limiting examples of biologically active agents include following: adrenergic blocking agents, anabolic agents, androgenic steroids, antacids, anti-asthmatic agents, anti-allergenic materials, anti-cholesterolemic and anti-lipid agents, anti-cholinergics and sympathomimetics, anti-coagulants, anti-convulsants, anti-diarrheal, anti-emetics, anti-hypertensive agents, anti-infective agents, anti-inflammatory agents such as steroids, non-steroidal anti-inflammatory agents, anti-malarials, anti-manic agents, anti-nauseants, anti-neoplastic agents, anti-obesity agents, anti-parkinsonian agents, anti-pyretic and analgesic agents, anti-spasmodic agents, anti-thrombotic agents, anti-uricemic agents, anti-anginal agents, antihistamines, anti-tussives, appetite suppressants, benzophenanthridine alkaloids, biologicals, cardioactive agents, cerebral dilators, coronary dilators, decongestants, diuretics, diagnostic agents, erythropoietic agents, estrogens, expectorants, gastrointestinal sedatives, agents, hyperglycemic agents, hypnotics, hypoglycemic agents, ion exchange resins, laxatives, mineral supplements, mitotics, mucolytic agents, growth factors, neuromuscular drugs, nutritional substances, peripheral vasodilators, progestational agents, prostaglandins, psychic energizers, psychotropics, sedatives, stimulants, thyroid and anti-thyroid agents, tranquilizers, uterine relaxants, vitamins, antigenic materials, and prodrugs.

Still further, the following listing of peptides, proteins, and other large molecules may also be used, such as interleukins 1 through 18, including mutants and analogues; interferons a, y, and which may be useful for cartilage regeneration, hormone releasing hormone (LHRH) and analogues, gonadotropin releasing hormone transforming growth factor (TGF); fibroblast growth factor (FGF); tumor necrosis factor-α); nerve growth factor (NGF); growth hormone releasing factor (GHRF), epidermal growth factor (EGF), connective tissue activated osteogenic factors, fibroblast growth factor homologous factor (FGFHF); hepatocyte growth factor (HGF); insulin growth factor (IGF); invasion inhibiting factor-2 (IIF -2); bone morphogenetic proteins 1-7 (BMP 1-7); somatostatin; thymosin-a-y-globulin; superoxide dismutase (SOD); and complement factors, and biologically active analogs, fragments, and derivatives of such factors, for example, growth factors.

Various forms of the biologically active agents may be used. These include, without limitation, such forms as uncharged molecules, molecular complexes, salts, ethers, esters, amides, prodrug forms and the like, which are biologically activated when implanted, injected or otherwise placed into a subject.

The charge, lipophilicity or hydrophilicity of a composition may be modified by employing an additive. For example, surfactants may be used to enhance miscibility of poorly miscible liquids. Examples of suitable surfactants include dextran, polysorbates and sodium lauryl sulfate. In general, surfactants are used in low concentrations, generally less than about 5%.

As used herein, the term “decreased PDE9 activity” means a manipulated decrease in the polypeptide activity of the PDE9 enzyme as a result of genetic disruption or manipulation of the PDE9 gene function that causes a reduction in the level of functional PDE9 polypeptide in a cell, or as the result of administration of a pharmacological agent that inhibits PDE9 activity.

As used herein, the term “subject” refers to any mammal, including, but not limited to, mammals of the order Rodentia, such as mice and hamsters, and mammals of the order Logomorpha, such as rabbits. It is preferred that the mammals are from the order Carnivora, including Felines (cats) and Canines (dogs). It is more preferred that the mammals are from the order Artiodactyla, including Bovines (cows) and Swines (pigs) or of the order Perssodactyla, including Equines (horses). It is most preferred that the mammals are of the order Primates, Ceboids, or Simoids (monkeys) or of the order Anthropoids (humans and apes). An especially preferred mammal is the human.

As used herein, the term “therapeutically effective” means resulting in a decrease in body fat or percentage of body fat, increase in lean mass or percentage of lean mass, decreased symptoms of HFpEF or other cardiac disease, decreased insulin resistance, increased glucose tolerance, reduced hyperlipidemia (total cholesterol lowering, triglyceride lowering), enhanced oxidative metabolism, and other improvements in obesity-related metabolic and functional defects in the heart, skeletal muscle, and other organs.

The PDE9 inhibitor compositions will be formulated, dosed and administered in a manner consistent with good medical practice. Factors for consideration in this context include the particular disorder being treated, the particular mammal being treated, the clinical condition of the individual patient, the cause of the disorder, the site of delivery of the inhibitor, the method of administration, the scheduling of administration, and other factors known to medical practitioners. The “therapeutically effective amount” of the inhibitor to be administered will be governed by such considerations, and can be the minimum amount necessary to prevent, ameliorate or treat a disorder of interest. As used herein, the term “effective amount” is an equivalent phrase refers to the amount of a therapy (e.g., a prophylactic or therapeutic agent), which is sufficient to reduce the severity and/or duration of a disease, ameliorate one or more symptoms thereof, prevent the advancement of a disease or cause regression of a disease, or which is sufficient to result in the prevention of the development, recurrence, onset, or progression of a disease or one or more symptoms thereof, or enhance or improve the prophylactic and/or therapeutic effect(s) of another therapy (e.g., another therapeutic agent) useful for treating a disease.

The PDE9 inhibitor compounds may be administered to a mammal at dosage levels in the range of from about 0.1 mg to about 3,000 mg per day. For a normal adult human having a body mass of about 70 kg, a dosage in the range of from about 0.01 mg to about 100 mg per kg body mass is typically sufficient, and preferably from about 0.1 mg to about 10 mg per kg. However, some variability in the general dosage range may be required depending upon the age and mass of the subject being treated, the intended route of administration, the particular compound being administered, and the like. The determination of dosage ranges and optimal dosages for a particular mammalian subject is within the ability of one of ordinary skill in the art having benefit of the instant disclosure.

According to the methods of the present invention, a PDE9 inhibitor, a stereoisomer or prodrug thereof, or a pharmaceutically acceptable salt of the PDE9 inhibitor, stereoisomer, or prodrug, may be administered in the form of a pharmaceutical composition comprising a pharmaceutically acceptable carrier, vehicle, or diluent. Accordingly, a PDE9 inhibitor, a stereoisomer or prodrug thereof, or a pharmaceutically acceptable salt of the PDE9 inhibitor, stereoisomer, or prodrug, may be administered to a subject separately or together in any conventional dosage forms, including, oral, buccal, sublingual, ocular, topical (e.g., transdermal), parenteral (e.g., intravenous, intramuscular, or subcutaneous), rectal, intracisternal, intravaginal, intraperitoneal, intravesical, local (e.g., powder, ointment, or drop), nasal and/or inhalation dosage forms.

In accordance with another embodiment, the present invention provides methods for increasing the percentage of lean body muscle in an estrogen deficient female subject comprising administering to the subject an effective amount of a phosphodiesterase-9 enzyme (PDE-9) inhibitor.

In accordance with another embodiment, the present invention provides methods for increasing the percentage of lean body muscle in a male subject with a disease or condition where the NO signaling pathway is compromised, comprising administering to the subject an effective amount of a phosphodiesterase-9 enzyme (PDE-9) inhibitor.

As used herein, the term “metabolic rate” is defined by the oxygen consumption per day normalized to body mass. As such, and increase in oxygen consumption translates to a higher metabolic rate. The normalization to body mass can be lean body mass only, or can be total body mass. The present inventors have used a model showing that fat contributes around 20% of the total, so essentially the calculation is normalized so the fat weight is less contributory but not ignored. With these calculations the inventors still saw greater oxygen consumed in the treated mice—indicating a higher metabolic rate.

In accordance with a further embodiment, the present invention provides methods for increasing the metabolic rate in an estrogen deficient female subject comprising administering to the subject an effective amount of a phosphodiesterase-9 enzyme (PDE-9) inhibitor.

In accordance with a further embodiment, the present invention provides methods for increasing the metabolic rate in a male subject in which nitric oxide signaling is deficient due to a disease associated with oxidative stress, comprising administering to the subject an effective amount of a phosphodiesterase-9 enzyme (PDE-9) inhibitor.

In accordance with an embodiment, the present invention provides methods for decreasing the body weight of an estrogen deficient female subject in which nitric oxide signaling is deficient, comprising administering to the subject an effective amount of a phosphodiesterase-9 enzyme (PDE-9) inhibitor.

In accordance with an embodiment, the present invention provides methods for decreasing the body weight of a male subject in which nitric oxide signaling is deficient due to a disease associated with oxidative stress, comprising administering to the subject an effective amount of a phosphodiesterase-9 enzyme (PDE-9) inhibitor.

In accordance with another embodiment, the present invention provides methods for decreasing cardiac hypertrophy in an obese estrogen deficient female subject in which nitric oxide signaling is deficient due to a disease associated with oxidative stress, comprising administering to the subject an effective amount of a phosphodiesterase-9 enzyme (PDE-9) inhibitor.

In accordance with another embodiment, the present invention provides methods for decreasing cardiac hypertrophy in an obese male subject in which nitric oxide signaling is deficient due to a disease associated with oxidative stress, comprising administering to the subject an effective amount of a phosphodiesterase-9 enzyme (PDE-9) inhibitor.

As used herein, the term “overweight” and the more severe “obese” conditions, in an adult person 18 years or older, constitute having greater than ideal body weight (more specifically, greater than ideal body fat) and are generally defined by body mass index (BMI), which is correlated with total body fat and the relative risk of suffering from premature death or disability due to disease as a consequence of the overweight or obese condition. The health risks increase with the increase in excessive body fat. BMI is calculated by weight in kilograms divided by height in meters squared (kg/m²) or, alternatively, by weight in pounds, multiplied by 703, divided by height in inches squared (lbs×703/in²). “Overweight” typically constitutes a BMI of between 25.0 and 29.9. “Obesity” is typically defined as a BMI of 30 or greater (see, e.g., National Heart, Lung, and Blood Institute, Clinical Guidelines on the Identification, Evaluation, and Treatment of Overweight and Obesity in Adults, The Evidence Report, Washington, DC: U.S. Department of Health and Human Services, NIH publication no. 98-4083, 1998). In heavily muscled individuals, the correlation between BMI, body fat, and disease risk is weaker than in other individuals. Therefore, assessment of whether such heavily muscled individuals are in fact overweight or obese may be more accurately performed by another measure such as direct measure of total body fat or waist-to-hip ratio assessment.

In accordance with a further embodiment, the present invention provides methods for improving cardiac function in an obese estrogen deficient female subject comprising administering to the subject an effective amount of a phosphodiesterase-9 enzyme (PDE-9) inhibitor.

As used herein, the term “improved or improving cardiac function” is defined by various measures of higher contractile performance including, for example, increased ejection fraction, and improved diastolic relaxation and filling.

In accordance with a further embodiment, the present invention provides methods for improving cardiac function in an obese male subject with a disease or condition where the NO signaling pathway is compromised, comprising administering to the subject an effective amount of a phosphodiesterase-9 enzyme (PDE-9) inhibitor.

As used herein, the term “metabolic syndrome”, and as according to the Adult Treatment Panel III (ATP III; National Institutes of Health: Third Report of the National Cholesterol Education Program Expert Panel on Detection, Evaluation, and Treatment of High Blood Cholesterol in Adults (Adult Treatment Panel III), Executive Summary; Bethesda, Md., National Institutes of Health, National Heart, Lung and Blood Institute, 2001 (NIH pub. no. 01-3670), occurs when a person has three or more of the following criteria:
1. Abdominal obesity: waist circumference >102 cm in men and >88 cm in women;
2. Hypertriglyceridemia: ≥50 mg/dl (1.695 mmol/l);
3. Low HDL cholesterol: <40 mg/di (1.036 mmol/l) in men and <50 mg/dl (1.295 mmol/l) in women;
4. High blood pressure: ≥30/85 mmHg;
5. High fasting glucose: ≥10 mg/dl (>6.1 mmol/l); or, as according to World Health Organization criteria (Alberti and Zimmet, Diabet. Med. 15: 539-53, 1998), when a person has diabetes, impaired glucose tolerance, impaired fasting glucose, or insulin resistance plus two or more of the following abnormalities:
1. High blood pressure: ≥60/90 mmHg;
2. Hyperlipidemia: triglyceride concentration ≥50 mg/dl (1.695 mmol/l) and/or HDL cholesterol <35 mg/dl (0.9 mmol/l in men and <39 mg/dl (1.0 mmol/l) in women;
3. Central obesity: waist-to-hip ratio of >0.90 for men and >0.85 in women and/or BMI >30 kg/m²;
4. Microalbuminuria: urinary albumin excretion rate ≥20 μg/min or an albumin-to-creatinine ratio ≥20 mg/kg.

In accordance with yet another embodiment, the present invention provides methods for improving glucose tolerance in an estrogen deficient female subject comprising administering to the subject an effective amount of a phosphodiesterase-9 enzyme (PDE-9) inhibitor.

As used herein, the term “improving glucose tolerance” means that a subject undergoing an oral glucose tolerance test, would have lower blood glucose levels, where normal is typically defined as lower than 140 mg/dL (7.8 mmol/L), and a blood glucose level between 140 and 199 mg/dL (7.8 and 11 mmol/L) is considered indicative of impaired glucose tolerance, or prediabetes.

In accordance with yet another embodiment, the present invention provides methods for improving glucose tolerance in an male subject with a disease or condition where the NO signaling pathway is compromised, comprising administering to the subject an effective amount of a phosphodiesterase-9 enzyme (PDE-9) inhibitor.

In accordance with still a further embodiment, the present invention provides methods for improving insulin sensitivity in an estrogen deficient female subject comprising administering to the subject an effective amount of a phosphodiesterase-9 enzyme (PDE-9) inhibitor.

In accordance with still a further embodiment, the present invention provides methods for improving insulin sensitivity in a male subject with a disease or condition where the NO signaling pathway is compromised, comprising administering to the subject an effective amount of a phosphodiesterase-9 enzyme (PDE-9) inhibitor.

By a “high fat diet”, as administered to a genetically-modified or wild type mouse, is meant a diet composed of at least 45% kcal fat, and, preferably, at least 58% fat. Exemplary diets include the Surwit diet (Surwit et al., Metabolism 47: 1354-1359; Surwit et al., Metabolism 47: 1089-1096, 1998; Surwit et al., J. Biol. Chem. 271: 9437-9440, 1996; and Surwit et al., Metabolism 44: 645-651, 1995), D12451 Rodent Diet (45% kcal fat, Research Diets, Inc., New Brunswick, N.J.), and 30 D12331 Rodent Diet (58% kcal fat, Research Diets, Inc.). Some nutrition information states that for humans, a high fat diet is one where the percentage of kcal from fat is between 50-75%. For a 2000 kcal diet, about 83 to 125 g per day.

The precise effective amount of the PDE9 inhibitor or inhibitors for a human subject will depend upon the severity of the subject's disease state, general health, age, weight, gender, diet, time and frequency of administration, drug combination(s), reaction sensitivities, and tolerance or response to therapy. A routine experimentation can determine this amount and is within the judgment of the medical professional. Compositions may be administered individually to a patient, or they may be administered in combination with other drugs, hormones, agents, and the like.

In some embodiments, the PDE9 inhibitors may be co-administered or sequentially administered to a subject with one or more additional biologically active agents. An active agent and a biologically active agent are used interchangeably herein to refer to a chemical or biological compound that induces a desired pharmacological and/or physiological effect, wherein the effect may be prophylactic or therapeutic. The terms also encompass pharmaceutically acceptable, pharmacologically active derivatives of those active agents specifically mentioned herein, including, but not limited to, salts, esters, amides, prodrugs, active metabolites, analogs and the like. When the terms “active agent,” “pharmacologically active agent” and “drug” are used, then, it is to be understood that the invention includes the active agent per se as well as pharmaceutically acceptable, pharmacologically active salts, esters, amides, prodrugs, metabolites, analogs etc. The active agent can be a biological entity, such as a virus or cell, whether naturally occurring or manipulated, such as transformed.

The following examples have been included to provide guidance to one of ordinary skill in the art for practicing representative embodiments of the presently disclosed subject matter. In light of the present disclosure and the general level of skill in the art, those of skill can appreciate that the following examples are intended to be exemplary only and that numerous changes, modifications, and alterations can be employed without departing from the scope of the presently disclosed subject matter. The synthetic descriptions and specific examples that follow are only intended for the purposes of illustration, and are not to be construed as limiting in any manner to make compounds of the disclosure by other methods.

### EXAMPLES

Our laboratory has studied the impact of PDE9 inhibition on the heart, in particular in male mice subjected to pressure overload stress to induced cardiac hypertrophy, dysfunction, and pathological fibrosis. Over the past 1.5 years, we have turned to females, and in particular added the impact of high fat diet induced obesity and metabolic syndrome. This is then combined with a mild pressure stress on the heart to stimulate production of natriuretic peptides and some hypertrophy of the heart muscle. We found in females under these conditions, that the PDE9 inhibitor can indeed ameliorate cardiac disease, similar to what was observed in males. However, there was minimal impact on peripheral body weight, metabolism, or fat mass in these studies.

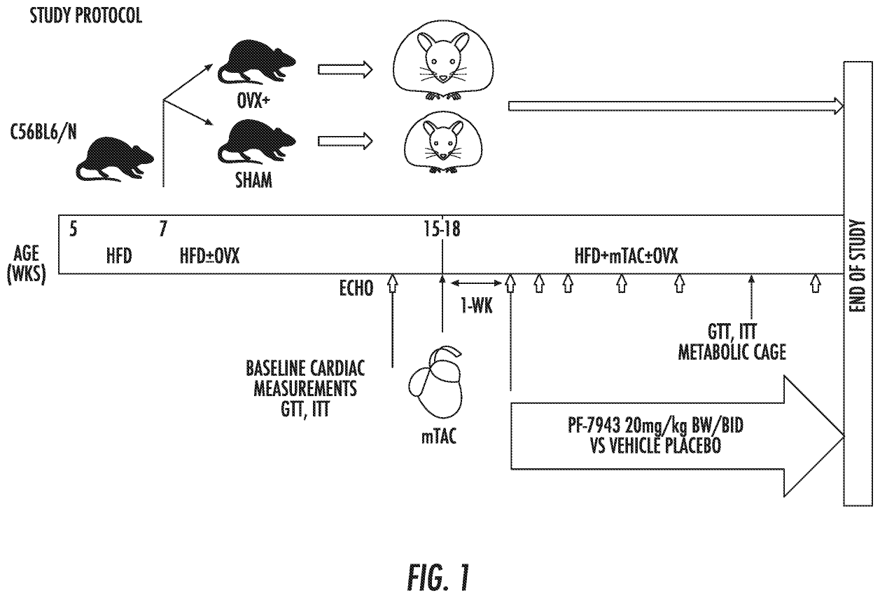

All animals were fed a high fat diet (HFD) consisting of 60% kcal derived from fat was fed to both male and female C57BL6N mice for 3 months. Mice were divided into two groups: 1) Controls with intact ovaries; 2) Ovariectomized (Ovx) mice. Mice were assigned to receive trans-aortic constriction (TAC) or sham surgery after 3 months of HFD, and further randomized to placebo vehicle and PDE9-I (PDE9 inhibitor oral gavage 20 mg/kg twice daily) (FIG. 1). Comparison of Group 1 and 2 tests the efficacy of PDE9-I to ameliorate and/or reverse HFD and pressure-overload pathophysiology independent of sex hormone status.

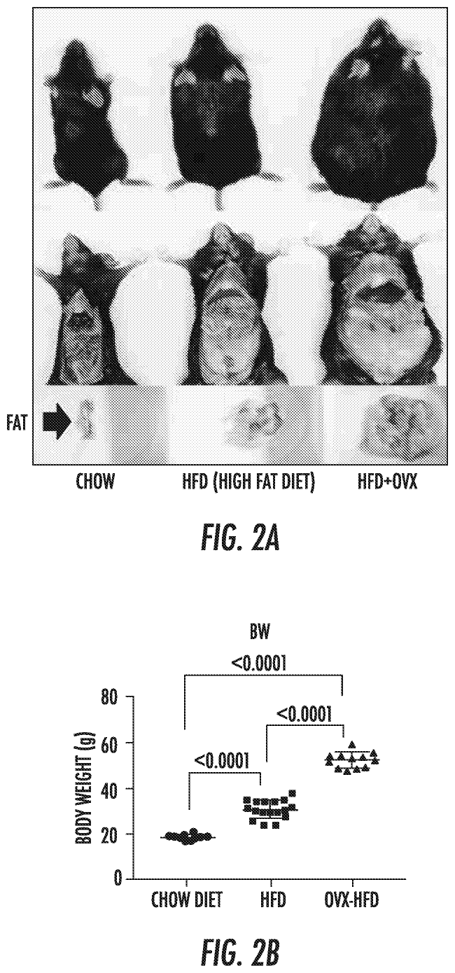

Generation of morbid obesity. The inventors used a somewhat different strain that is more widely used for these studies—specifically the C57BL/6N strain (most common is C57BL/6J). The difference is that the 6N mice develop greater weight gain on a HFD, and in particular, female mice do. In addition, when the ovaries of the females were removed, and then placed on the same HFD, the weight gain is profound (FIG. 2A). Normal females on this background strain will weigh around 20 g, when fed a HFD alone, their average weight is 35 g; however the combination of a HFD and ovariectomy results in weights between 42-45 g (FIG. 2B). This is a morbid obesity equivalent.

Changes in cardiac function and structure were measured using echocardiogram, histopathology and markers of cardiac stress (Nppa Gene expression by RT-qPCR). For qPCR, RNA is isolated from left ventricular myocardium (Trizol Reagent, Invitrogen), reverse transcribed to cDNA (High Capacity RNA-to-cDNA Kit, Applied Biosystems, Life Technologies), and undergoes PCR amplification using TaqMan probes for atrial natriuretic peptide (Nppa) (mouse #Mm01255747_g1, rat #Rn00664637_g1). The threshold cycle value was determined using the crossing point method, and samples normalized to the GAPDH for each run. Echocardiography was performed in conscious mice using serial M-mode transthoracic echocardiography (VisualSonics Vevo 2100, 18-38 MHz transducer; SanoSite Inc.). Images were obtained and analyzed by an individual blinded to the animal condition.

Whole body metabolism: Indirect calorimetry was used to assess metabolic parameters in live animals using the OxyMAX Comprehensive Lab Animal Monitoring System (Columbus Instruments, Columbus Ohio). Basic metabolic rate (VO₂ consumption), VCO₂ release and energy expenditure (EE) were measured by indirect calorimetry in high-fat-fed OVX mice groups treated with (Drug) and without PDE9 inhibitor (Control). Respiratory exchange ratio (RER) was calculated from VCO₂ release-to-VO₂ consumption ratio (VCO₂ release/VO₂ consumption). CLAMS cabinet was used to measure oxygen consumption rate at 22° C. (VO₂), CO₂ emission, respiratory exchange ratio, heat production, food consumption (g consumed per day), and total and ambulatory activity per day.

Glucose (GTT) and insulin (ITT) Tolerance Tests assessment was done to measure peripheral tissue glucose and insulin sensitivity. In brief, GTTs were conducted by fasting mice overnight. The next day glucose (1 g/kg body weight) was bolus administered intraperitoneally into awake mice. Blood samples were taken at 10, 20, 30, 60, 90 and 120 minutes following injection for measurement of plasma glucose concentrations. Impairment of glucose tolerance indicates problems with glucose homeostasis.

For ITTs, mice were fasted for 2 hours, and insulin (0.5 U/kg body weight) was administered intraperitoneally into awake mice. Blood samples were taken at 10, 20, 30, 60, 90 and 120 minutes following injection for measurement of plasma glucose concentrations. The degree to which glucose falls following the insulin bolus is indicative of whole-body insulin action.

Body composition: total fat mass, lean mass, and water content was determined by quantitative nuclear magnetic resonance (Echo-MRI®). In brief, after 20 weeks on the HFD, total body fat and lean body mass were assessed non-invasively by EchoMRI instrument. The analyzer delivers precise body composition measurements of fat, lean, free water, and total water masses in live animals weighing up to 100 grams. Conscious mice were placed in a constraint tube that was then inserted into the EchoMRI for a period of approximately 30 s and these measurements obtained.

As demonstrated in the data in this patent, we have now performed studies in female mice lacking ovaries (and thus estrogen), then placed on a high fat diet (60% fat) to induce severe obesity, and then further stimulated with a low level of high pressure stress on the heart to induce mild hypertrophy and activate natriuretic peptide signaling. Unlike females on the same diet and heart stress but with their ovaries, those without ovaries demonstrate marked weight loss from PDE9 inhibition, in combination with improvement in their metabolic signature (reduced fasting glucose, cholesterol, and triglycerides). There is no change in food intake, nor change in activity, so the mechanism appears to engage intrinsic modulation of the fat intake. We see some benefit in male mice lacking estrogen, though the magnitude is substantially less than we observe in the ovariectomized females. The current invention—for using PDE9 inhibitors to treat obesity and associated co-morbidities including the heart in post-menopausal women (natural and iatrogenic) was not anticipated by any existing literature and or even provision patent filings. Indeed, the failure of any publications subsequent to 2003 to address the issue of PDE9 inhibition and obesity at all is a reflection of the lack of meaningful findings prior to our discovery and the present inventive methods. The discovery was only possible once we understood the preserved if not enhanced efficacy of PDE9 inhibition in conditions where the NO-GC signaling pathway is compromised. In addition, we believe this inhibition will be most effective in conditions in which there is some enhanced synthesis of natriuretic peptide, due to the existence of cardiac disease. This makes it particularly suitable for HFpEF.

Without being held to any particular theory, the inventors also believe PDE9 inhibition will be effective in disorders where the NO signaling pathway is compromised as this transfers additional physiological importance to the natriuretic peptide-signaling pathway that PDE9 regulates. In addition to women lacking estrogen (post-menopause), we include conditions that stimulate oxidative stress that also compromise the NO-signaling pathway.

FIG. 1 displays the protocol for the primary experiment. Female mice (C57BL/6N) were placed on a 60% fat diet starting at age 5-weeks. Two weeks later, there were randomized to receive bilateral oophorectomy or sham surgery. Cardiac imaging for ventricular function and morphometry was determined 2 months later. Between 8-11 weeks after ovariectomy, the mice underwent a second surgical procedure introducing a mild aortic constriction (TAC) in the transverse aorta to increase pressure load on the heart. The purpose of the TAC procedure was to stimulate the heart to synthesize natriuretic peptide, which is important for the generation of cyclic GMP which PDE9 regulates. Serial echocardiograms were obtained, and starting 1 week after TAC, mice were further randomized to receive vehicle control or a PDE9 inhibitor (PF-7943). Intact mouse metabolic studies, fat/lean mass determinations, and heart function were assessed. At terminal study, tissues were harvested for molecular signaling.

Female mice on this high fat diet showed a 50% increase in total body mass, associated with visceral adiposity. Those that further underwent ovariectomy display >100% increase in body mass, virtually all of it due to fat. (FIG. 2A, 2B).

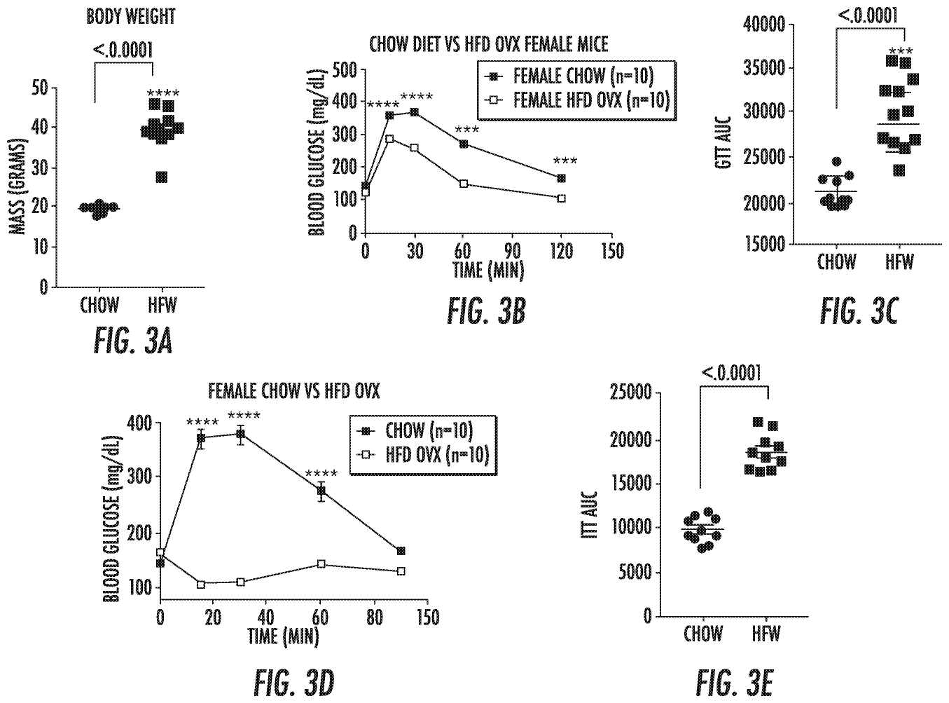

Ovariectomized HFD mice were compared to non-ovariectomized mice on normal chow—assessing metabolic status by glucose tolerance and insulin tolerance testing (FIG. 3A-C). Mice were fasted overnight, and the following morning, a bolus of glucose (1 g/kg body weight) was administered intraperitoneally into awake mice. Blood samples were taken at 10, 20, 30, 60, 90 and 120 min following injection for measurement of plasma glucose concentrations. The results show impaired glucose uptake as both the peak and integral of the glucose-time curve were greater in the OVX+HFD mice. Summary results for area under the curve (AUC) are shown. Panel D shows insulin tolerance test (ITT) results, and summary data for AUC in Panel. Here, mice were fasted for 2 hrs, and insulin (0.5 U/kg body weight) administered i.p. Blood sampling was as for the GTT. The extent of glucose decline is a measure of insulin sensitivity. The OVX-HFD mice shows less decline. Together these results indicate marked metabolic deficits typical of type 2 diabetes mellitus.

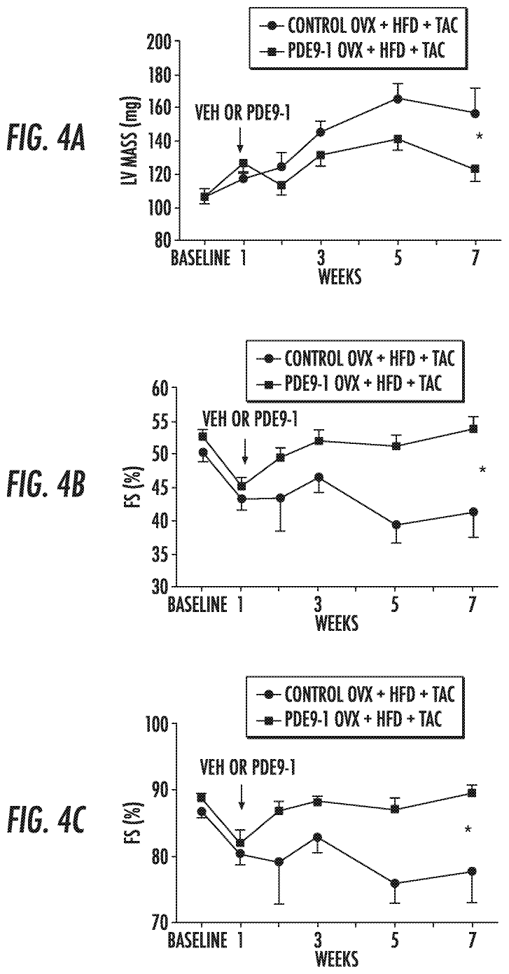

Cardiac function measured throughout this protocol demonstrates similar declines in cardiac systolic function (fractional shortening FS) and ejection fraction (EF), and a small rise in left ventricular hypertrophy (LV mass) after 1 week following TAC in each treatment group. (FIG. 4A-4C) Active drug was initiated after this point, and the mice receiving the PDE9 inhibitor show reduced LV mass, and improved FS and EF. These disparities were significantly different for each variable (p<0.05 for drug×time interaction by covariance analysis).

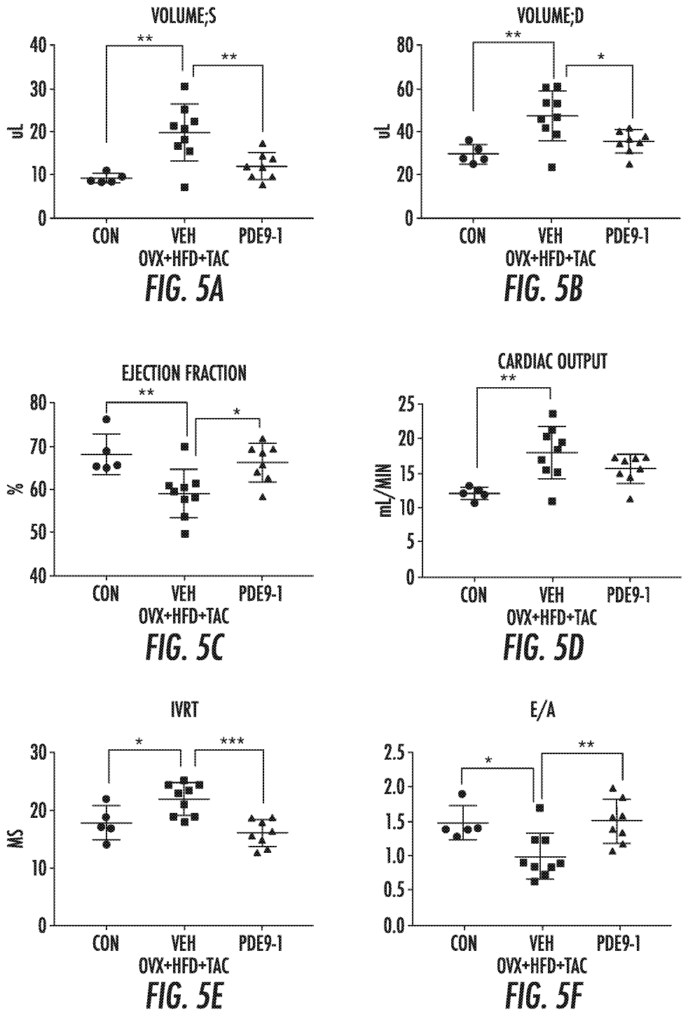

Cardiac function in both systole and diastole were assessed at end-of the study using echocardiography. FIG. 5 shows summary results for the OVX+TAC mice on HFD with or without PDE9 inhibitor treatment. Control mice on normal chow is also displayed for comparison. Compared to both control mice and OVX+HFD+TAC treated with the PDE9 inhibitor, OVX+HFD+TAC mice on vehicle treatment show evidence of cardiac disease reflected by larger left ventricle volumes both at end-systolic (volume, s) and end-diastole (volume,d) and a lower ejection fraction. Cardiac output is greater in these mice consistent with greater body mass. Isovolumic ventricular relaxation time (IVRT) was longer and ratio of early to late ventricular filling (E/A) was shorter in these mice on vehicle (both consistent with diastolic dysfunction), and this is restored by treatment with the PDE9 inhibitor.

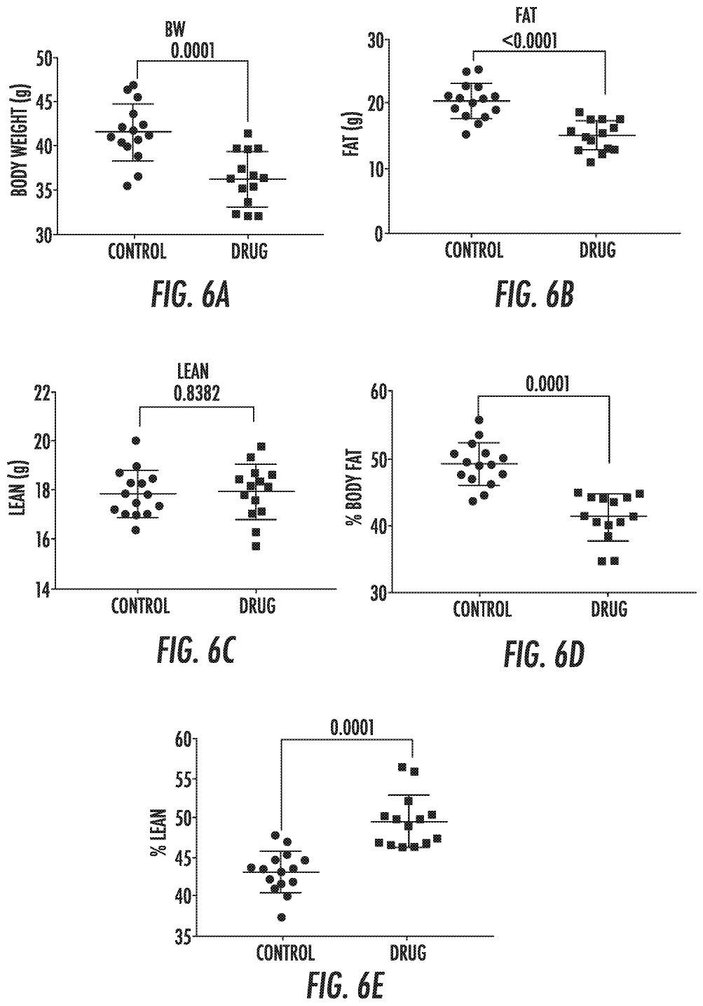

Body composition analysis is shown in FIG. 6. After 20 weeks on the HFD+ 8 wks mTAC, total body fat and lean body mass were assessed non-invasively by EchoMRI. The figure shows PDE9 inhibition reduced fat mass without altering lean body mass, so the percentage of total fat declined, while percent of lean mass increased.

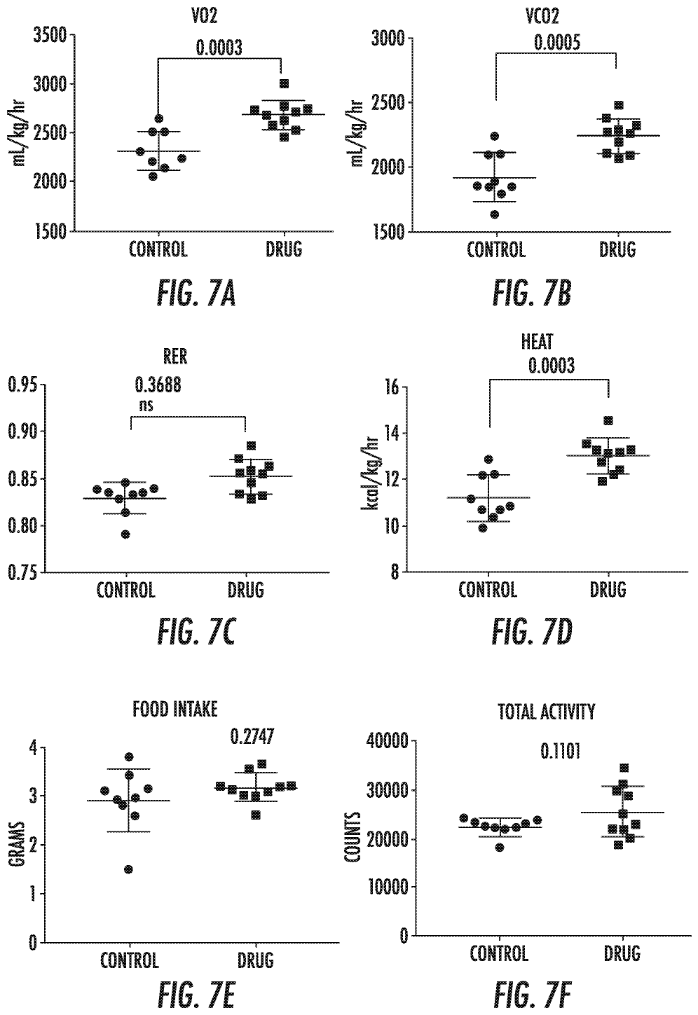

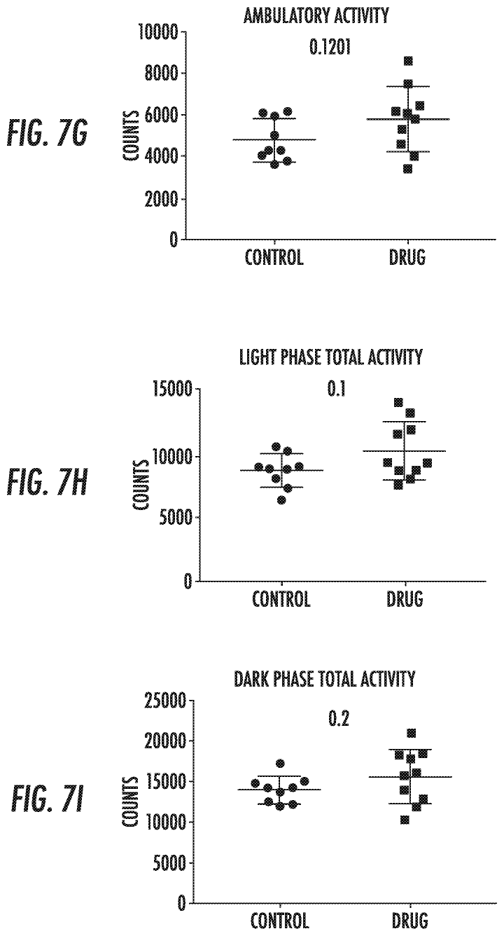

FIG. 7 shows the effects of PDE9 inhibition on basal metabolic rate (total body oxygen consumption—VO2), carbon dioxide release, VCO2, respiratory exchange ratio (RER), and estimated energy expenditure (heart) measured by indirect calorimetry in high-fat-fed OVX mice groups treated with PDE9-inhibitor (Drug) or vehicle control (Control). RER is calculated from VCO2/VO2 ratio. The results show PDE9I therapy increases total oxygen consumption normalized to body mass, and CO₂ generation also normalized to total body mass. Even if fat mass is assumed to contribute only 15% of total metabolic activity relative to lean mass, these differences remain significant (data not shown). Measures of activity and food intake showed no significant differences between vehicle and PDE9-I treatment groups. Activity is shown during the dark cycle (most active for mice) and light cycle, and for ambulatory activity. P values are for unpaired t-tests between groups.

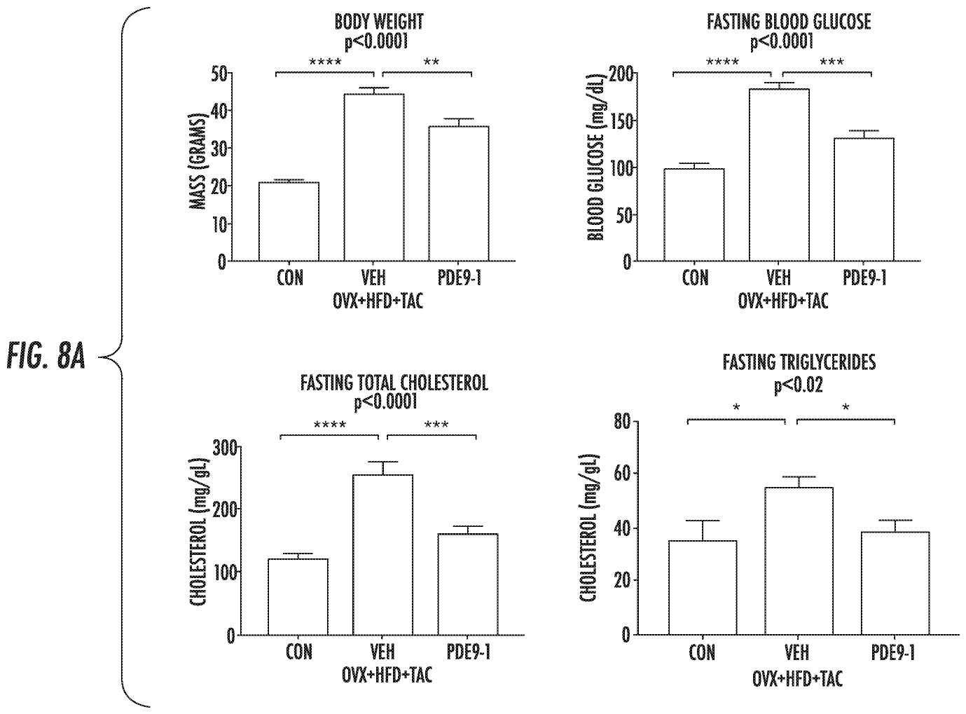

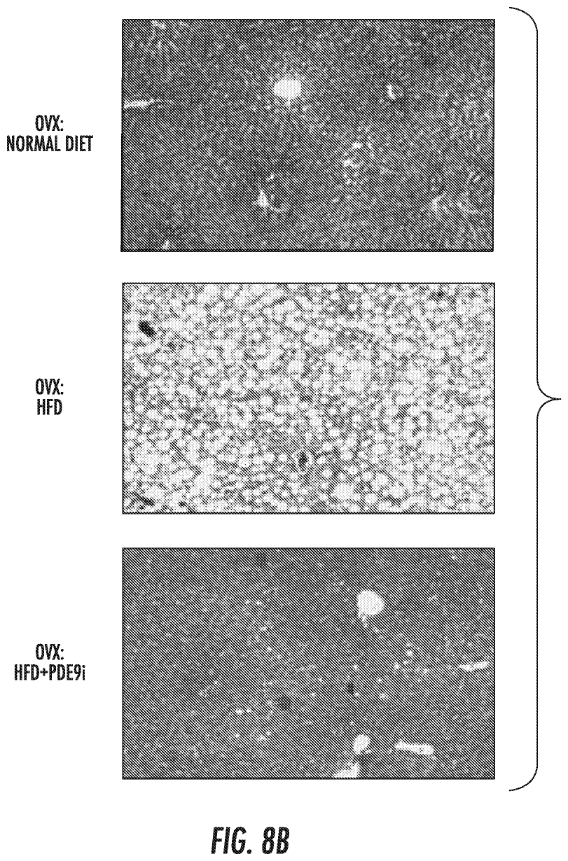

Multiple biomarkers of metabolic syndrome and fat accumulation in the liver were improved in OVX+HFD+TAC female mice by PDE9 inhibition (FIG. 8). Data are shown at end-of study for fasting blood glucose, cholesterol, triglycerides, and histology of the liver. All blood markers are significantly increased in the OVX+HFD+TAC obesity model compared to normal diet controls, and this is reduced by active PDE9-I therapy. The liver displays marked fat accumulation in the OVX+HFD+TAC obesity model, and this is markedly reduced by active PDE9-I therapy.

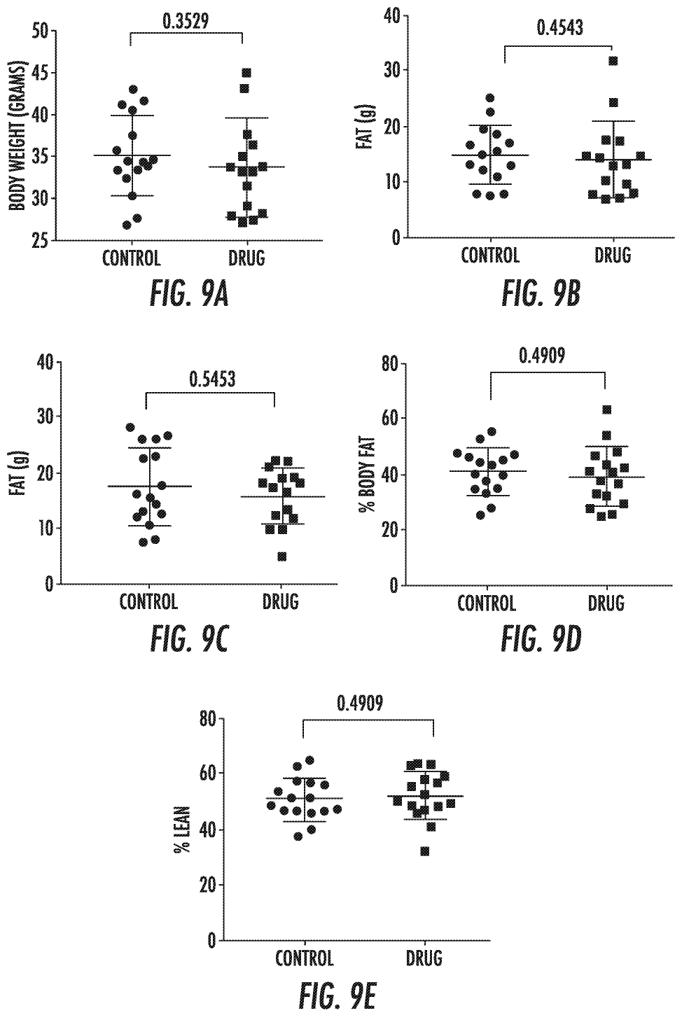

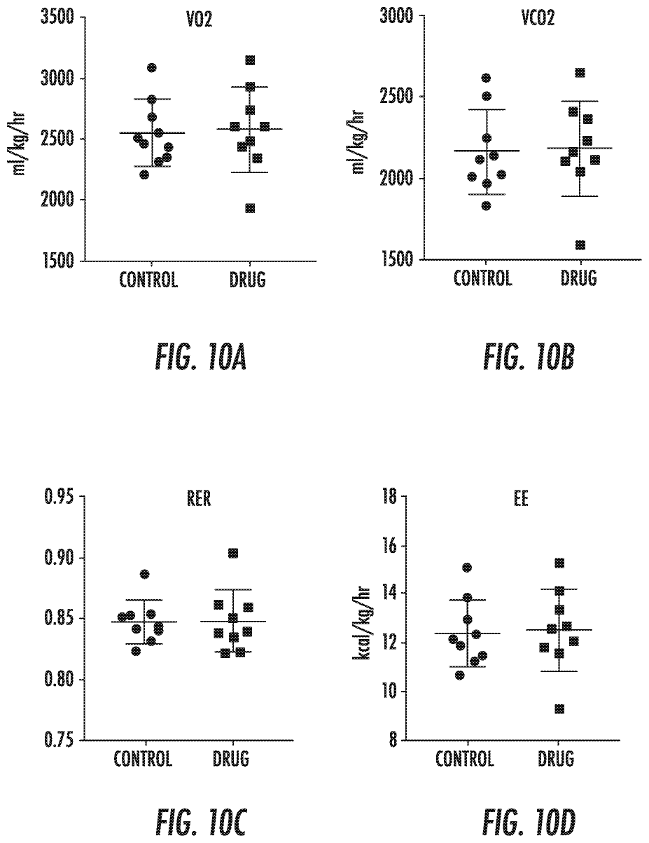

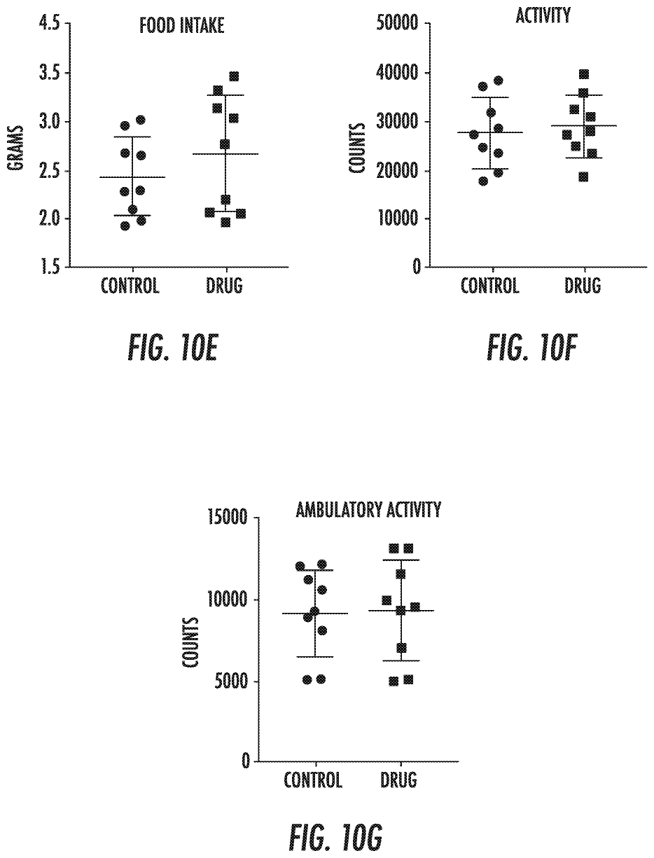

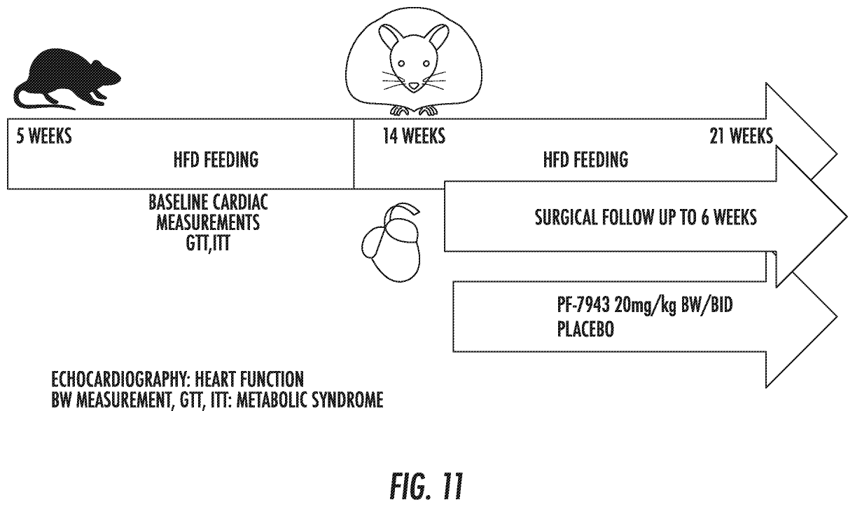

In a second experiment, we examined the impact of PDE9 inhibition on female mice (C57BL6/N) subjected to HFD+TAC, but without ovariectomy and thus had normal estrogen levels. As shown in FIG. 9, there was no significant change in total body weight, total fat mass and lean mass, and thus percent fat or lean tissue as a consequence of receiving active PDE0-I therapy. As shown in FIG. 10, there was no difference in total animal oxygen consumption, CO₂ production, respiratory exchange ratio, activity, or food intake as a consequence of administration of active PDE9-I therapy. This experiment shows the importance of having a decline in estrogen in females as a key contributor to the efficacy of a PDE9-inhibitor to reduce obesity.

In a third experiment, we exposed male mice of a similar starting age (5 weeks) to the same HFD protocol, and then same TAC protocol. As before, mice were randomized to either vehicle control or active PDE9-inhibitor starting 1 week after the TAC procedure. FIG. 11.

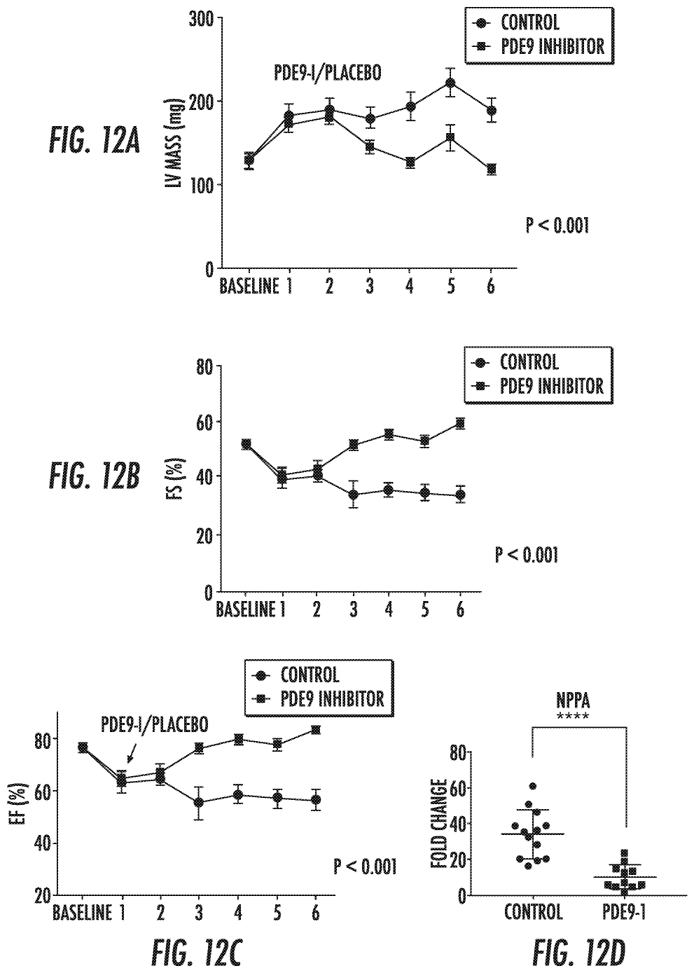

Male mice treated with the PDE9-inhibitor displayed significant improvement of left ventricular heart function (increases in both fractional shortening and ejection fraction) and reduced left heart mass (hypertrophy) similar to what had been observed in the OVX+HFD+TAC females (FIG. 12A-C). Molecular signaling of hypertrophy was indexed by the gene expression of Nppa, a biomarker of cardiac stress (FIG. 12D). This was reduced by active therapy.

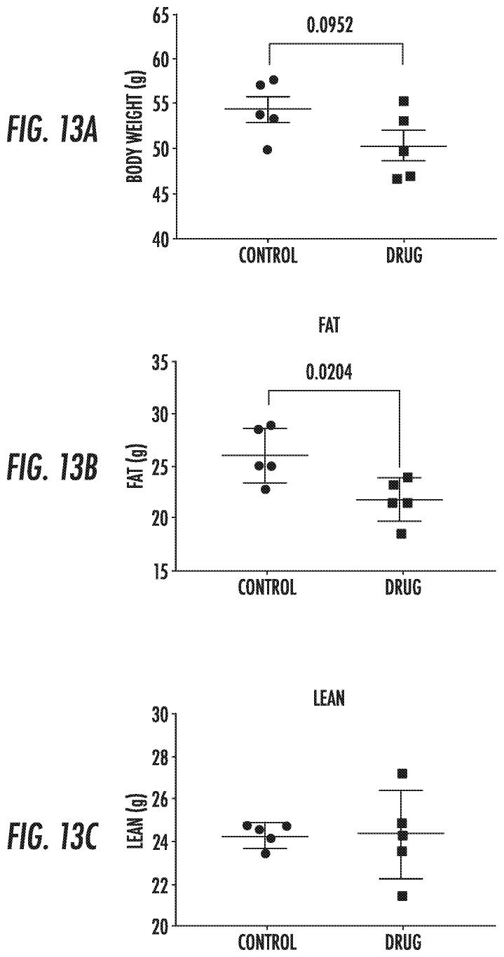

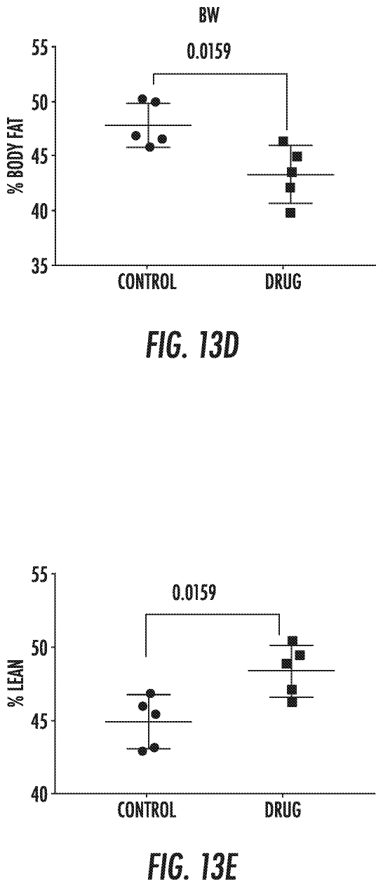

Unlike normal female mice on HFD+TAC, males on a HFD+TAC showed a significant reduction in total fat mass, though total body mass was only borderline altered (FIG. 13A-13E) The relative percent decline in total fat was more modest than observed in the OVX+HFD+TAC female model.

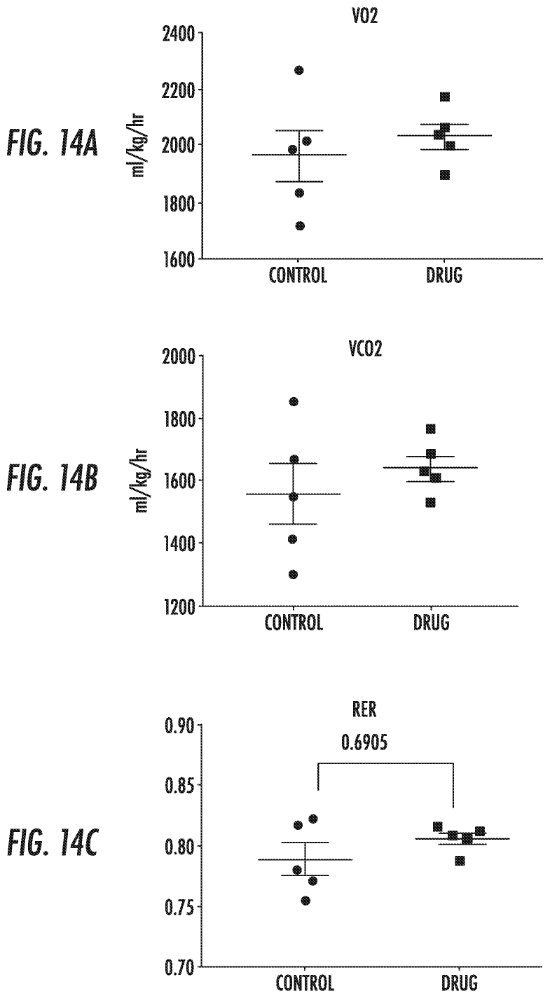

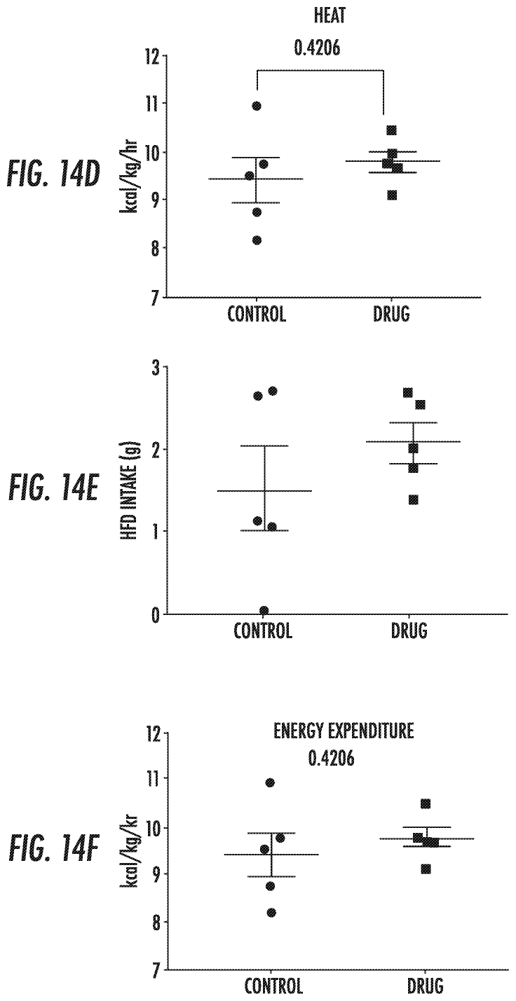

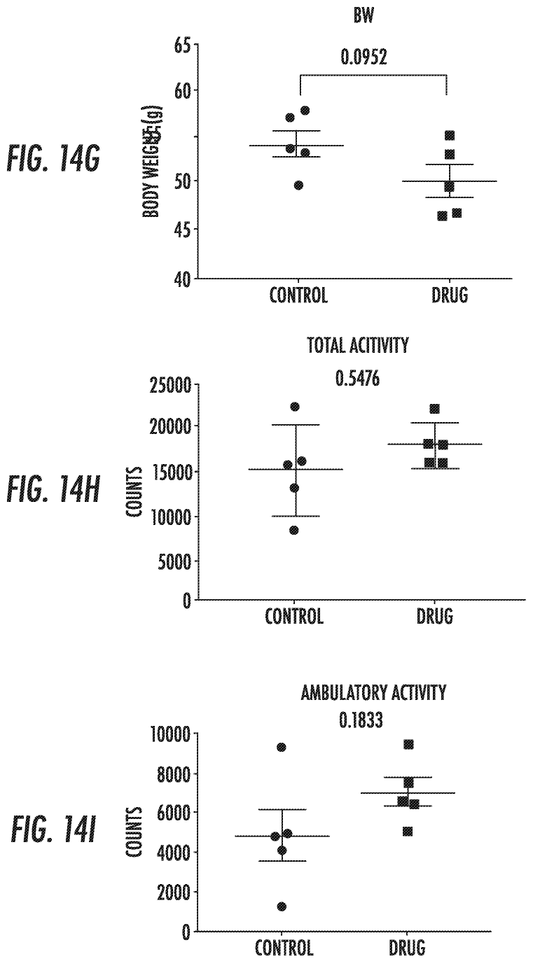

Metabolic studies in male mice on HFD did not demonstrate increases in metabolic rates and estimates of heat generation and energy expenditure (FIG. 14A-14G). Thus, while males do demonstrate marked improvement in cardiac function and mass, and some decline in total fat mass, the magnitude of metabolic changes are less than observed in ovariectomized females on the same HFD and subjected to the same mild pressure load.

Taken together these three studies show: 1) females with estrogen deficiency that are subjected to diet induced obesity and mild cardiac pressure stress show a marked improvement in cardiac function, metabolic activity, and reduced obesity associated with reversal of metabolic syndrome defects from a PDE9 inhibitor. 2) females with normal estrogen levels show little impact from the same inhibitor, supporting an importance of reducing the signaling pathway that couples to nitric oxide (e.g. via estrogen in females). 3) males who do not have significant estrogen levels but do have a well-functioning nitric oxide signaling pathway display a somewhat intermediate response, with improved heart function, and some decline in fat mass, but less total metabolic changes. These results support the new invention regarding the use of PDE9 inhibitors to treat obesity, revealing a new class of subjects for which this is effective. They also likely explain the failure of any prior study to demonstrate efficacy of PDE9 inhibition for obesity, since it was first cloned in 1998, as no one had examined OVX females.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. Use of a phosphodiesterase-9 enzyme (PDE-9) inhibitor in an effective amount for decreasing the percentage of body fat in an obese subject in which estrogen is deficient.

2. The use of claim 1, wherein the subject is male.

3. The use of claim 1, wherein the subject is female.

4. The use of claim 3 wherein the estrogen deficiency is due to menopause.

5. The use of claim 3 wherein the estrogen deficiency is due to surgical means.

6. The use of claim 5, wherein the estrogen deficiency is due to oophorectomy.

7. The use of any of claims 1-6, wherein the subject has a disease associated with oxidative stress.

8. The use of any of claims 1-7, wherein the disease associated with oxidative stress is selected from the group consisting of: aging, metabolic syndromes including obesity and type 1 and 2-diabetes, vascular disorders including hypertension, atherosclerosis, stiffening of the arteries, inflammatory diseases including viral, bacterial, and protozoal infections; autoimmune diseases including rheumatological disorders and inflammatory bowel disease, environmental pollutants, smoking, and other disorders.

9. Use of a phosphodiesterase-9 enzyme (PDE-9) inhibitor in an effective amount for decreasing the percentage of body fat in an obese subject in which nitric oxide signaling is deficient.

10. The use of claim 9, wherein the subject is male.

11. The use of claim 9, wherein the subject is female.

12. The use of any of claims 9-11, wherein the subject has a disease associated with oxidative stress.

13. The use of any of claims 9-12, wherein the disease associated with oxidative stress is selected from the group consisting of: aging, metabolic syndromes including obesity and type 1 and 2-diabetes, vascular disorders including hypertension, atherosclerosis, stiffening of the arteries, inflammatory diseases including viral, bacterial, and protozoal infections; autoimmune diseases including rheumatological disorders and inflammatory bowel disease, environmental pollutants, smoking, and other disorders.

14. Use of a phosphodiesterase-9 enzyme (PDE-9) inhibitor in an effective amount for increasing the percentage of lean muscle mass in a subject in which estrogen is deficient.

15. The use of claim 14, wherein the subject is male.

16. The use of claim 14, wherein the subject is female.

17. The use of claim 16 wherein the estrogen deficiency is due to menopause.

18. The use of claim 16 wherein the estrogen deficiency is due to surgical means.

19. The use of claim 18, wherein the estrogen deficiency is due to oophorectomy.

20. The use of any of claims 14-19, wherein the subject has a disease associated with oxidative stress.

21. The use of any of claims 14-20, wherein the disease associated with oxidative stress is selected from the group consisting of: aging, metabolic syndromes including obesity and type 1 and 2-diabetes, vascular disorders including hypertension, atherosclerosis, stiffening of the arteries, inflammatory diseases including viral, bacterial, and protozoal infections; autoimmune diseases including rheumatological disorders and inflammatory bowel disease, environmental pollutants, smoking, and other disorders.

22. Use of a phosphodiesterase-9 enzyme (PDE-9) inhibitor in an effective amount for increasing the percentage of lean muscle mass in a subject in which nitric oxide signaling is deficient.

23. The use of claim 22, wherein the subject is male.

24. The use of claim 22 wherein the subject is female.

25. The use of any of claims 22-24, wherein the subject has a disease associated with oxidative stress.

26. The use of any of claims 22-25, wherein the disease associated with oxidative stress is selected from the group consisting of: aging, metabolic syndromes including obesity and type 1 and 2-diabetes, vascular disorders including hypertension, atherosclerosis, stiffening of the arteries, inflammatory diseases including viral, bacterial, and protozoal infections; autoimmune diseases including rheumatological disorders and inflammatory bowel disease, environmental pollutants, smoking, and other disorders.

27. Use of a phosphodiesterase-9 enzyme (PDE-9) inhibitor in an effective amount for increasing the metabolic rate in a subject in which estrogen is deficient.

28. The use of claim 27, wherein the subject is male.

29. The use of claim 27, wherein the subject is female.

30. The use of claim 29 wherein the estrogen deficiency is due to menopause.

31. The use of claim 29 wherein the estrogen deficiency is due to surgical means.

32. The use of claim 31, wherein the estrogen deficiency is due to oophorectomy.

33. The use of any of claims 27-32, wherein the subject has a disease associated with oxidative stress.

34. The use of any of claims 27-33, wherein the disease associated with oxidative stress is selected from the group consisting of: aging, metabolic syndromes including obesity and type 1 and 2-diabetes, vascular disorders including hypertension, atherosclerosis, stiffening of the arteries, inflammatory diseases including viral, bacterial, and protozoal infections; autoimmune diseases including rheumatological disorders and inflammatory bowel disease, environmental pollutants, smoking, and other disorders.

35. Use of a phosphodiesterase-9 enzyme (PDE-9) inhibitor in an effective amount for increasing the metabolic rate in a subject in which nitric oxide signaling is deficient.

36. The use of claim 35, wherein the subject is male.

37. The use of claim 35, wherein the subject is female.

38. The use of any of claims 35-37, wherein the subject has a disease associated with oxidative stress.

39. The use of any of claims 35-38, wherein the disease associated with oxidative stress is selected from the group consisting of: aging, metabolic syndromes including obesity and type 1 and 2-diabetes, vascular disorders including hypertension, atherosclerosis, stiffening of the arteries, inflammatory diseases including viral, bacterial, and protozoal infections; autoimmune diseases including rheumatological disorders and inflammatory bowel disease, environmental pollutants, smoking, and other disorders.

40. Use of a phosphodiesterase-9 enzyme (PDE-9) inhibitor in an effective amount for decreasing cardiac hypertrophy in a subject in which estrogen is deficient.

41. The use of claim 40, wherein the subject is male.

42. The use of claim 40, wherein the subject is female.

43. The use of claim 42 wherein the estrogen deficiency is due to menopause.

44. The use of claim 42 wherein the estrogen deficiency is due to surgical means.

45. The use of claim 44, wherein the estrogen deficiency is due to oophorectomy.

46. The use of any of claims 40-45, wherein the subject has a disease associated with oxidative stress.

47. The use of any of claims 40-46, wherein the disease associated with oxidative stress is selected from the group consisting of: aging, metabolic syndromes including obesity and type 1 and 2-diabetes, vascular disorders including hypertension, atherosclerosis, stiffening of the arteries, inflammatory diseases including viral, bacterial, and protozoal infections; autoimmune diseases including rheumatological disorders and inflammatory bowel disease, environmental pollutants, smoking, and other disorders.

48. Use of a phosphodiesterase-9 enzyme (PDE-9) inhibitor in an effective amount for decreasing cardiac hypertrophy in a subject in which nitric oxide signaling is deficient.

49. The use of claim 48, wherein the subject is male.

50. The use of claim 48, wherein the subject is female.

51. The use of any of claims 48-50, wherein the subject has a disease associated with oxidative stress.

52. The use of any of claims 48-51, wherein the disease associated with oxidative stress is selected from the group consisting of: aging, metabolic syndromes including obesity and type 1 and 2-diabetes, vascular disorders including hypertension, atherosclerosis, stiffening of the arteries, inflammatory diseases including viral, bacterial, and protozoal infections; autoimmune diseases including rheumatological disorders and inflammatory bowel disease, environmental pollutants, smoking, and other disorders.

53. Use of a phosphodiesterase-9 enzyme (PDE-9) inhibitor in an effective amount for improving cardiac function in a subject in which estrogen is deficient.

54. The use of claim 53, wherein the subject is male.

55. The use of claim 53, wherein the subject is female.

56. The use of claim 55 wherein the estrogen deficiency is due to menopause.

57. The use of claim 55 wherein the estrogen deficiency is due to surgical means.

58. The use of claim 57, wherein the estrogen deficiency is due to oophorectomy.

59. The use of any of claims 53-58, wherein the subject has a disease associated with oxidative stress.

60. The use of any of claims 53-59, wherein the disease associated with oxidative stress is selected from the group consisting of: aging, metabolic syndromes including obesity and type 1 and 2-diabetes, vascular disorders including hypertension, atherosclerosis, stiffening of the arteries, inflammatory diseases including viral, bacterial, and protozoal infections; autoimmune diseases including rheumatological disorders and inflammatory bowel disease, environmental pollutants, smoking, and other disorders.

61. Use of a phosphodiesterase-9 enzyme (PDE-9) inhibitor in an effective amount for improving cardiac function in a subject in which nitric oxide signaling is deficient.

62. The use of claim 61, wherein the subject is male.

63. The use of claim 61, wherein the subject is female.

64. The use of any of claims 61-63, wherein the subject has a disease associated with oxidative stress.

65. The use of any of claims 61-64, wherein the disease associated with oxidative stress is selected from the group consisting of: aging, metabolic syndromes including obesity and type 1 and 2-diabetes, vascular disorders including hypertension, atherosclerosis, stiffening of the arteries, inflammatory diseases including viral, bacterial, and protozoal infections; autoimmune diseases including rheumatological disorders and inflammatory bowel disease, environmental pollutants, smoking, and other disorders.
